SEQUENCE LISTING (34 sequences)

SEQ ID NO: 1 - protein, 6 aa, Artificial
  peptide
WQRHGI

SEQ ID NO: 2 - protein, 6 aa, Artificial
  peptide
MWRGWQ

SEQ ID NO: 3 - protein, 6 aa, Artificial
  peptide
RHLGWF

SEQ ID NO: 4 - protein, 6 aa, Artificial
  peptide
GWLHQR

SEQ ID NO: 5 - protein, 6 aa, Artificial
  peptide
MWRAWQ

SEQ ID NO: 6 - protein, 5 aa, Artificial
  peptide
MWRWQ

SEQ ID NO: 7 - protein, 6 aa, Artificial
  peptide
MWRGFQ

SEQ ID NO: 8 - protein, 6 aa, Artificial
  peptide
GWRGWQ

SEQ ID NO: 9 - protein, 6 aa, Artificial
  peptide
WQRHGL

SEQ ID NO: 10 - protein, 6 aa, Artificial
  peptide
WQRHGV

SEQ ID NO: 11 - protein, 6 aa, Artificial
  peptide
WQRHAI

SEQ ID NO: 12 - protein, 6 aa, Artificial
  peptide
WNRHGI

SEQ ID NO: 13 - protein, 6 aa, Artificial
  peptide
RMWGWN

SEQ ID NO: 14 - protein, 6 aa, Artificial
  peptide
WHRLQG

SEQ ID NO: 15 - protein, 6 aa, Artificial
  peptide
WHRGQL

SEQ ID NO: 16 - protein, 6 aa, Artificial
  peptide
HWRGWW

SEQ ID NO: 17 - protein, 6 aa, Artificial
  peptide
HWRGLQ

SEQ ID NO: 18 - protein, 6 aa, Artificial
  peptide
HWRGWV

SEQ ID NO: 19 - protein, 7 aa, Artificial
  peptide
GWLHWQR

SEQ ID NO: 20 - protein, 6 aa, Artificial
  peptide
  MISC_FEATURE (3)..(3): wherein Xaa is citrulline
HWXGWV

SEQ ID NO: 21 - protein, 6 aa, Artificial
  peptide
MFRGWQ

SEQ ID NO: 22 - protein, 6 aa, Artificial
  peptide
MWRGWN

SEQ ID NO: 23 - protein, 6 aa, Artificial
  peptide
  MISC_FEATURE (1)..(1): wherein Xaa is nor-leucine
XWRGWQ

SEQ ID NO: 24 - protein, 6 aa, Artificial
  peptide
MGRGWQ

SEQ ID NO: 25 - protein, 6 aa, Artificial
  peptide
  MISC_FEATURE (3)..(3): wherein Xaa is citrulline
MWXGWQ

SEQ ID NO: 26 - protein, 6 aa, Artificial
  peptide
MWRGGQ

SEQ ID NO: 27 - protein, 6 aa, Artificial
  peptide
  MISC_FEATURE (3)..(3): wherein Xaa is citrulline
WQXHGI

SEQ ID NO: 28 - protein, 6 aa, Artificial
  peptide
WQRAGI

SEQ ID NO: 29 - protein, 6 aa, Artificial
  peptide
FQRHGI

SEQ ID NO: 30 - protein, 7 aa, Artificial
  peptide
WQRHGIC

SEQ ID NO: 31 - protein, 7 aa, Artificial
  peptide
MWRGWQC

SEQ ID NO: 32 - protein, 7 aa, Artificial
  peptide
WQRGHIC

SEQ ID NO: 33 - protein, 5 aa, Artificial
  peptide
WQRHI

SEQ ID NO: 34 - protein, 7 aa, Artificial
  peptide
HWRGWVC

(19) United States
(12) Patent Application Publication
(10) Pub. No.: US 2023/0399358 A1
(43) Pub. Date: Dec. 14, 2023

(54) IMMUNOGLOBULIN PURIFICATION PEPTIDES AND THEIR USE

(71) Applicant: North Carolina State University, Raleigh, NC (US)

(72) Inventors: Stefano Menegatti, Raleigh, NC (US); Xingqing Xiao, Apex, NC (US); Carol K. Hall, Raleigh, NC (US); Hannah Reese, Raleigh, NC (US)

(21) Appl. No.: 17/766,884

(22) PCT Filed: Oct. 8, 2020
(86) PCT No.: PCT/US2020/054669

§ 371 Date: Apr. 6, 2022

Related U.S. Application Data
(60) Provisional application No. 62/912,118, filed on Oct. 8, 2019.

(51) Int. Cl. C07K 1/22; C07K 7/06

(52) U.S. Cl. CPC C07K 1/22; C07K 7/06

(57) ABSTRACT

The present invention provides synthetic peptides comprising an amino acid sequence of any one of SEQ ID NOs: 1-17 or an amino acid sequence having at least 80%, 85%, 90%, or 95% sequence identity to the amino acid sequence of any one of SEQ ID NOs:1-17. Also described herein are solid supports including peptides and methods of using such peptides and solid supports.

### STATEMENT OF GOVERNMENT SUPPORT

This invention was made with government support under grant number 1830272 awarded by the National Science Foundation. The government has certain rights in the invention.

### FIELD OF THE INVENTION

The present invention relates to synthetic peptides having an amino acid sequence of any one of SEQ ID NOs: 1-17 or an amino acid sequence having at least 80% sequence identity to the amino acid sequence of any one of SEQ ID NOs:1-17, and methods of using the same.

### BACKGROUND OF THE INVENTION

Monoclonal antibodies (“mAbs”) form the backbone of several current therapeutic strategies, including as treatment for cancer and immunological disorders. Therapeutic mAbs are extremely expensive to develop and produce. The technology for the purification of therapeutic mAbs in current platform biomanufacturing processes relies on Protein A adsorbents to achieve simultaneous purification and concentration during the product capture step. Owing to its high affinity for mAbs—most frequently belonging to the IgG1 and IgG4 subclasses—Protein A-based purification affords a log removal value (LRV) of host cell protein (HCP) of ˜2.5-3.0 (Shukla et al. 2008 Biotechnology Progress 24(3):615-622). Despite these advantages, Protein A adsorbents exhibit several significant limitations. They are expensive (up to $15,000 per liter), suffer from limited biochemical stability in cleaning conditions or in the presence of feed-stock proteolytic enzymes, elution must be carried out at low pH, and they cannot capture any putative IgG3 therapeutics (Hober et al. 207 J. of Chromatography B: Analytical Technologies in the Biomedical and Life Sciences 848:40-47; Leblebici et al. 2014 J. of Chromatography B: Analytical Technologies in the Biomedical and Life Sciences 962:89-93). Protein A fragments and aggregated mAbs are highly toxic and immunogenic, so their potential release into the product stream must be closely monitored. Surmounting challenges associated with Protein A media is one of the main drivers of innovation in bioseparation technology. In this context, synthetic alternatives to protein ligands have been, and still are, thoroughly scrutinized.

In an effort to manufacture adsorbents with no batch-to-batch variability, fewer immunogenic and pathogenic components, milder elution conditions, and lower cost, many synthetic ligands have been investigated. Mixed mode ligands (MMLs), which combine the ionic and charge interactions of ion exchange chromatography (IEC) with attraction to non-polar elements found in hydrophobic interaction chromatography (HIC), are cheap to produce and have been extensively investigated (Tong et al. 2016 J. of Chromatography A 1429:258-264; Holstein et al. 2012 J. of Chromatography A 1233:152-155). Several MMLs, such as triazine based MAbSorbent A1P and A2P, MEP Hypercel, CaptoAdhere, and CaptoMMC have become commercially available and are often used in MAb polishing steps. However, MMLs lack the mAb binding affinity and selectivity of affinity ligands like Protein A, and thus are not suitable for capture.

The present invention overcomes shortcomings in the art by providing synthetic peptide ligands and methods of using the same, optionally in purification and/or detection of an immunoglobulin and/or fragment thereof, e.g., as peptide mimetics of Protein A.

### SUMMARY OF THE INVENTION

One aspect of the present invention is directed to a synthetic peptide having an amino acid sequence of any one of SEQ ID NOs:1-17 or an amino acid sequence having at least 80% sequence identity to the amino acid sequence of any one of SEQ ID NOs:1-17. The peptide may have a host cell protein (HCP) logarithmic removal value (LRV) of at least 2.0, 2.1, 2.2., 2.3, 2.4, 2.5, 2.6, 2.7, 2.8, 2.9, 3.0, 3.1, 3.2, 3.3, 3.4, 3.5, or more as measured by a HCP-specific ELISA assay, optionally wherein the peptide has a HCP LRV of at least 2.5. In some embodiments, the peptide binds an immunoglobulin (e.g., IgG) or fragment thereof, optionally wherein the peptide binds the Fc portion of the immunoglobulin or fragment thereof.

Another aspect of the present invention is directed to an article comprising a solid support (e.g., a resin) and a peptide as described herein. The peptide may be covalently bound to the solid support. In some embodiments, the article is an affinity adsorbent.

A further aspect of the present invention is directed to a method of detecting an immunoglobulin or fragment thereof present in a sample, the method comprising: contacting the sample and a peptide as described herein and/or an article as described herein under suitable conditions wherein the peptide binds the immunoglobulin or fragment thereof to provide a peptide-bound immunoglobulin; and detecting the peptide, thereby detecting the immunoglobulin or fragment thereof.

Another aspect of the present invention is directed to a method of purifying an immunoglobulin or fragment thereof present in a sample, comprising: contacting the sample and a peptide as described herein and/or an article as described herein under suitable conditions wherein the peptide binds the immunoglobulin or fragment thereof to provide a peptide-bound immunoglobulin; and separating (e.g., releasing, eluting, etc.) the immunoglobulin or fragment thereof from the peptide and/or article, thereby purifying the immunoglobulin or fragment thereof from the sample.

These and other aspects of the invention are addressed in more detail in the description of the invention set forth below.

### BRIEF DESCRIPTION OF THE DRAWINGS

FIG. 1 shows the binding sites as predicted by MD simulation using the AMBER 15 package. Binding complexes of sequences in diagram (A) WQRHGI (SEQ ID NO:1), diagram (B) HWRGWV (SEQ ID NO:18), diagram (C) MWRGWQ (SEQ ID NO:2), diagram (D) RHLGWF (SEQ ID NO:3), and diagram (E) GWLHQR (SEQ ID NO:4) with CH2 subunit of human IgG (PDB ID: 1FCC) are pictured.

FIGS. 2A-2D show contributions of individual peptide residues to the binding energy for the human IgG Fc fragment were obtained using the implicit-solvent MM/GBSA approach with the variable internal dielectric constant model for (FIG. 2A) WQRHGI (SEQ ID NO:1), (FIG. 2B) MWRGWQ (SEQ ID NO:2), (FIG. 2C) RHLGWF (SEQ ID NO:3), and (FIG. 2D) GWLHWQR (SEQ ID NO:19).

FIG. 3A shows a diagram of construction of Peptide-WB resin by (i) nucleophilic substitution of the native bromoalkyl functionality with an alkyl-amine spacer arm [-*-], (ii) activation with iodoacetic acid, and (iii) conjugation of the peptide ligand.

FIG. 3B shows ITC analysis of IgG:ligand binding at 25° C. Raw titration data for WQRHGI (SEQ ID NO:1) was integrated and peak area normalized to the molar amount of ligand added to the IGG solution. Data were fit using an independent binding model. The molar ratio denotes the ratio of ligand to protein. An effective K_{D} of 5.88×10⁻⁵ M was found using ITC.

FIGS. 4A-4B show binding isotherms of IgG on (FIG. 4A) MWRGWQC (SEQ ID NO:31)—WorkBeads and (FIG. 4B) WQRGHIC (SEQ ID NO:32)—WorkBeads.

FIG. 5 panels A-D show breakthrough curves of IgG on adsorbent WQRHGIC (SEQ ID NO:30)—WorkBeads at residence times of (panel A) 2 min and (panel B) 5 min, and adsorbent MWRGWQC (SEQ ID NO:31)—WorkBeads at residence times of (panel C) 2 min and (panel D) 5 min.

FIGS. 6A-6B show SDS-PAGE analysis (reducing conditions, Coomassie staining) of chromatographic fractions obtains from the purification of IgG from a CHO cell culture supernatant using the peptide ligands (FIG. 6A) MWRGWQ (SEQ ID NO:2) and RHLGWF (SEQ ID NO:3) and (FIG. 6B) WQRHGI (SEQ ID NO:1) and GWLHQR (SEQ ID NO:4). HWRGWV (SEQ ID NO:18) was used as a positive control. MW, molecular weight ladder; FT, flow-through; El1, first elution at pH4; El2, second elution at pH 2.8; IgG HC, IgG heavy chain; IgG LC, IgG light chain.

FIG. 7A shows Chromatograms obtained by injecting 0.5 mL of feedstock (human polyclonal IgG spiked in CHO-S cell culture supernatant) on 0.1 mL of either WQRHGI (SEQ ID NO:1)—WorkBeads or MWRGWQ (SEQ ID NO:2)—WorkBeads resins. Labels: FT, flow-through in PBS, pH 7.4; W, wash in 0.1 M NaCl in PBS, pH 7.4; EL, elution in 0.2 M sodium acetate, pH 4; R, regeneration in 0.1 M Glycine, pH 2.5.

FIG. 7B shows SDS-PAGE analysis (reducing conditions, silver staining) of chromatographic fractions obtained from the purification of IgG from a CHO cell culture supernatant using WQRHGI (SEQ ID NO:1)-WB resin. Labels: MW, molecular weight ladder; FT, flow-through; E, first elution at pH 4; R, second elution at pH 2.5; IgG HC, IgG heavy chain; IgG LC, IgG light chain.

FIG. 8 shows SDS-PAGE analysis (reducing conditions, silver staining) of chromatographic fractions obtained from the purification of IgG from a CHO cell culture supernatant using WQRHGI (SEQ ID NO:1)-WB resin. Labels: MW, molecular weight ladder; FT, flow-through; E, first elution at pH 4; R, second elution at pH 2.5; CHO proteins; Ld., Loaded protein; IgG HC, IgG heavy chain; IgG LC, IgG light chain.

FIG. 9 shows chromatograms obtained by successive injections of 0.5 mL of feedstock (human polyclonal IgG spiked in CHO-S cell culture supernatant) on 0.1 mL WQRHGI (SEQ ID NO:1)-WB resin at a 5 minute residence time. Resins were washed in PBS, eluted in 0.2 M sodium acetate, pH 4, and regenerated in 0.1 M Glycine, pH 2.5. In between runs, columns were cleaned with 1% acetic acid.

### DETAILED DESCRIPTION OF THE INVENTION

The present subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All publications, patent applications, patents, accession numbers and other references mentioned herein are incorporated by reference herein in their entirety.

While the following terms are believed to be well understood by one of ordinary skill in the art, the following definitions are set forth to facilitate explanation of the presently disclosed subject matter.

Following long-standing patent law convention, the terms “a” and “an” and “the” can mean one or more than one when used in this application, including the claims.

The use of any and all examples, or exemplary language (e.g., “such as”) provided herein, is intended merely to better illuminate the invention and does not pose a limitation on the scope of the invention unless otherwise claimed.

The term “and/or” when used in describing two or more items or conditions refers to situations where all named items or conditions are present or applicable, or to situations wherein only one (or less than all) of the items or conditions is present or applicable. Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of the length of a polypeptide sequence, dose, time, temperature, and the like, is meant to encompass variations of ±20%, ±10%, ±5%, ±1%, ±0.5%, or even ±0.1% of the specified amount.

As used herein, the term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting essentially of,” and “consisting of,” where one of these three terms is used herein, the presently disclosed subject matter can include the use of any of the other terms.

An “amino acid”, or “residue”, as used herein is defined as a molecule comprising an amino group, a carboxyl group, and a side chain functional group (R). When these R groups are appended to a backbone carbon on the “residue”, it is called a peptide, whereas attaching an R group to the amide nitrogen is a peptoid. Along with the position of the R-group along the polyamide chain (i.e. peptides and peptoids), another variation to the typical peptide backbone is the addition of one or more methylene units between the a carbon and amide nitrogen. These added carbons, called the (3-carbon (one additional methylene unit), y-carbon (two additional methylene units), or additional (6, etc.) carbons are also considered “amino acids” or “residues.” Examples of these residues can be seen in Tables 1A-1C.

TABLE 1A
Peptide and peptoid residues.

| Type | α |
|---|---|
| Peptides | 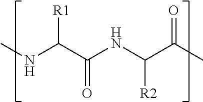 |
| Peptoids | 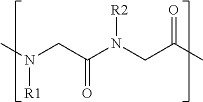 |

TABLE 1B
Peptide and peptoid residues.

| Type | β |
|---|---|
| Peptides | 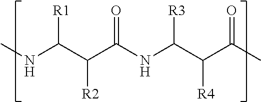 |
| Peptoids | 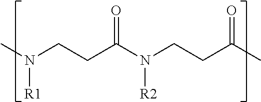 |

TABLE 1C
Peptide and peptoid residues.

| Type | γ |
|---|---|
| Peptides | 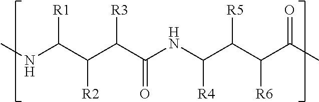 |
| Peptoids | 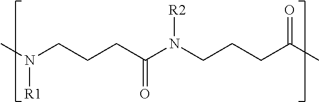 |

A “natural amino acid”, or “proteinogenic amino acid”, or “natural residue”, or “proteinogenic residue”, or “canonical amino acid”, or “canonical residue”, as used herein is defined as one of the following amino acids: alanine, citrulline, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine.

A “non-natural amino acid”, or “non-proteinogenic amino acid”, or “non-natural residue”, or “non-proteinogenic residue”, or “non-canonical amino acid”, or “non-canonical residue”, as used herein is defined as an amino acid whose side chain functional group (R) is different from those featured by the natural amino acids.

A non-proteinogenic, or non-natural or non-canonical, functional group (R) as used herein may be any suitable group or substituent, including but not limited to H, linear and cyclic alkyl, alkenyl, and alkynyl, possibly substituted and/or functionalized with functional groups such as alkoxy, mercapto, azido, cyano, carboxyl, hydroxyl, nitro, aryloxy, alkylthio, amino, alkylamino, arylalkylamino, substituted amino, acylamino, acyloxy, ester, thioester, carbamoyl, carboxylic thioester, ether, thioether, amide, amidino, sulfate, sulfoxyl, sulfonyl, sulfonyl, sulfonic acid, sulfonamide, urea, alkoxylacylamino, aminoacyloxy, keto, imine, nitrile, phosphate, thiol, amidine, oxime, nitrile, diazo, etc., these terms including combinations of these groups as discussed further below.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” (e.g., 80% sequence identity) refers to the percentage of identical amino acids in a linear polypeptide sequence of a reference (e.g., “query”) polypeptide as compared to another polypeptide when the two sequences are optimally aligned.

“Alkyl” as used herein alone or as part of another group, refers to a straight, branched chain, or cyclic, saturated or unsaturated, hydrocarbon containing from 1 or 2 to 10 or 20 carbon atoms, or more. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “akyl” or “loweralkyl” is intended to include both substituted and unsubstituted alkyl or loweralkyl unless otherwise indicated and these groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3. Alkyl may be saturated or unsaturated and hence the term “alkyl” as used herein is inclusive of alkenyl and alkynyl when the alkyl substituent contains one or more unsaturated bond (for example, one or two double or triple bonds). The alkyl group may optionally contain one or more heteroatoms (e.g., one, two, or three or more heteroatoms independently selected from O, S, and NR′, where R′ is any suitable substituent such as described immediately above for alkyl substituents), to form a linear heteroalkyl or heterocyclic group as specifically described below.

“Alkenyl” as used herein refers to an alkyl group as described above containing at least one double bond between two carbon atoms therein.

“Alkynyl” as used herein refers to an alkyl group as described above containing at least one triple bond between two carbon atoms therein.

“Alkylene” as used herein refers to an alkyl group as described above, with one terminal hydrogen removed to form a bivalent substituent.

“Heterocyclic group” or “heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and lower alkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” as used herein is as described in connection with heterocyclo above.

“Alkoxy” as used herein alone or as part of another group, refers to an alkyl or loweralkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Halo” as used herein refers to any suitable halogen, including fluorine, chlorine, bromine, and iodine.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical or a —N(Rₐ)C(O)R_{b} radical, where Rₐ and R_{b} are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl” as used herein refers to a compound of the formula —S(O)(O)R, where R is any suitable substituent such as alkyl, cycloalkyl, alkenyl, alkynyl or aryl.

“Sulfonate” as used herein refers to a compound of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)₂NRₐR_{b} radical, where Rₐ and R_{b} are any suitable substituent such as H, alkyl, cycloalkyl, alkenyl, alkynyl or aryl.

“Urea” as used herein alone or as part of another group refers to an —N(R_{c})C(O)NRₐR_{b} radical, where Rₐ, R_{b} and R_{c} are any suitable substituent such as H, alkyl, cycloalkyl, alkenyl, alkynyl or aryl.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl.

“Solid support” as used herein may comprise any suitable material, including natural materials (e.g., agarose and sepharose) either virgin or chemically modified (e.g., crosslinked), synthetic organic materials (e.g., organic polymers such as polymethacrylate or polyethylene glycol), metals and metal oxides (e.g., titanium, titania, zirconium and zirconia), inorganic materials (e.g., silica), and composites thereof. A solid support may be in any suitable shape or form including, but not limited to, a film, a receptacle such as a microtiter plate well (e.g., floors and/or walls thereof), a channel such as in a microfluidic device, a porous or non-porous particle (e.g., a bead formed from natural or synthetic polymers, inorganic materials such as glass or silica, membranes and non-woven membranes, and composites thereof, etc.) such as for chromatography column packings, a fiber, a microparticle, a nanoparticle (e.g., a magnetic nanoparticle), etc. In some embodiments, a solid support is a chromatographic resin, a membrane, a biosensor, a microbead, a magnetic bead, a paramagnetic particle, a quantum dot, and/or a microplate. In some embodiments, a solid support is a chromatographic resin such as, but not limited to, a sepharose-based resin (e.g., WORKBEADS™ resin), a poly-methacrylate-based resin (e.g., TOYOPEARL® resin), a silica-based resin, alumina, titania, or a glass-based resin.

“Linking group” as used herein may be any suitable reactive group, e.g., an alkene, alkyne, alcohol, azido, thiol, selenyl, phosphono, carboxylic acid, formyl, halide or amine group. A linking group may be displayed directly by the parent molecule (e.g., peptide) or by means of an intervening linker group (e.g., an aliphatic, aromatic, or mixed aliphatic/aromatic group such as an alkyl, aryl, arylalkyl, or alkylarylalkyl group, etc.). In some embodiments, a linking group may be an amino acid or a portion thereof (e.g., a side chain group of the amino acid). For example, in some embodiments, a linking group may be a cysteine and/or a thiol of a cysteine and/or a lysine and/or an amine of a lysine.

A peptide of the present invention may be prepared in accordance with known techniques including, but not limited to, those described in U.S. 2016/0075734 and/or U.S. Pat. No. 10,266,566.

The terms “antibody” and “immunoglobulin” include antibodies or immunoglobulins of any isotype, fragments of antibodies that retain specific binding to an antigen (e.g., Fab, Fv, single chain Fv (scFv), Fc fragments and Fd fragments), chimeric antibodies, humanized antibodies, single-chain antibodies, and fusion proteins including a portion of an antibody and a non-antibody protein. Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986. The antibodies can, in some embodiments, be detectably labeled, e.g., with a radioisotope, an enzyme which generates a detectable product, a fluorescent protein, a synthetic fluorescent molecule, and the like. The antibodies can in some embodiments be further conjugated to other moieties, such as members of specific binding pairs, e.g., biotin or avidin (members of the biotin-avidin specific binding pair), and the like. Also encompassed by the terms are Fab′, Fv, F(ab′)₂, and other antibody fragments that retain specific binding to antigen (e.g., any antibody fragment that comprises at least one paratope). As used herein, the term “Fc fragment” includes any protein or compound comprising an Fc portion of an immunoglobulin, e.g., an Fc-fusion protein.

As used herein, the term “host cell protein” (HCP) refers to any endogenous cell proteins of an organism (e.g., bacterial, mammalian, or avian) other than the desired target (e.g., immunoglobulin or fragment thereof). Thus, in a method of the present invention, a HCP is an endogenous protein that is a non-desired off-target and/or impurity. HCPs may be naturally inclusive in a sample (e.g., a cell culture fluid (e.g., supernatant), a plant extract, and/or bodily fluid) or may be isolated and/or purified HCPs present in a sample.

As used herein, the terms “logarithmic reduction” (LR) and “logarithmic reduction value” (LRV) refer to measurement of reduction of a contaminant (e.g., decontamination) and/or impurity in a process and/or method, e.g., a method of the present invention. The LRV is defined as the common logarithm of the ratio of the concentration of contaminant (e.g., non-desired off-target proteins, e.g., host cell protein (HCP)) before and after use of a purification method, wherein an increment of 1 corresponds to a reduction in concentration by a factor of 10. Thus, a 1-log reduction (i.e., LRV=1.0) equates to a 90% reduction of the contaminant concentration prior to the applied method, a 2-log reduction (i.e., LRV=2.0) corresponds to a 99% reduction, etc.

As used herein, the term “dissociation constant” or “K_{D}” in regard to a target-ligand complex refers to the ratio between the free target and the ligand-bound target. Specifically, the dissociation constant is an equilibrium constant that expresses the propensity of the target to bind reversibly to the ligand. The smaller the dissociation constant, the stronger the interaction is between the target and ligand. In some embodiments, the target is a protein and the ligand is a peptide such as a peptide of the present invention, which can form a complex with the target (e.g., protein).

Provided according to embodiments of the present invention are synthetic peptides. A peptide of the present invention comprises an amino acid sequence that has at least 80%, 85%, 90%, 91%, 92%, 93%, 94%, 95%, 96%, 97%, 98%, 99%, or 100% sequence identity to the amino acid sequence of any one of SEQ ID NOs:1-17. In some embodiments, a peptide of the present invention has an amino acid sequence of any one of SEQ ID NOs:1-17. In some embodiments, the peptide has an amino acid sequence of WQRHGI (SEQ ID NO:1), MWRGWQ (SEQ ID NO:2), RHLGWF (SEQ ID NO:3), GWLHQR (SEQ ID NO:4), MWRAWQ (SEQ ID NO:5), MWRWQ (SEQ ID NO:6), MWRGFQ (SEQ ID NO:7), GWRGWQ (SEQ ID NO:8), WQRHGL (SEQ ID NO:9), WQRHGV (SEQ ID NO:10), WQRHAI (SEQ ID NO:11), WNRHGI (SEQ ID NO:12), RMWGWN (SEQ ID NO:13) WHRLQG (SEQ ID NO:14), WHRGQL (SEQ ID NO:15), HWRGWW (SEQ ID NO:16), or HWRGLQ (SEQ ID NO:17). In some embodiments, a peptide of the present invention (e.g., a peptide having an amino acid sequence of any one of SEQ ID NOs:1-17) comprises a linking amino acid residue (e.g., a cysteine residue or a lysine residue) at the N-terminus and/or C-terminus optionally as the N-terminal amino acid residue and/or the C-terminal amino acid residue, respectively. A linking amino acid residue (e.g., a cysteine residue or lysine residue) may be used to attach (e.g., conjugate) the peptide to a solid support as the side chain group of the linking amino acid residue may react with a moiety of the solid support to create a covalent bond. For example, for a cysteine residue, reaction of the thiol of the cysteine residue with a moiety (e.g., epoxide, alkyl halide, maleimide, etc.) of the solid support may be used to attach the peptide to the solid support; or, for a lysine residue, reaction of the primary amine of the lysine residue with a moiety (e.g., epoxide, alkyl halide, N-hydroxysuccinimide ester, etc.) of the solid support may be used to attach the peptide to the solid support. In some embodiments, a peptide having an amino acid sequence of any one of SEQ ID NOs:1-17 comprises a cysteine residue as the C-terminal amino acid residue and the cysteine residue may be used to attach the peptide to a solid support.

A peptide of the present invention may have, provide and/or be configured to provide a host cell protein (HCP) logarithmic removal value (LRV) of at least 2 or more (e.g., about 2.0, 2.1, 2.2., 2.3, 2.4, 2.5, 2.6, 2.7, 2.8, 2.9, 3.0, 3.1, 3.2, 3.3, 3.4, 3.5, or more) as measured by a HCP-specific ELISA assay and/or a quantitative proteomic profile by mass spectrometry on chromatographic fractions from a separation performed on a representative cell culture fluid (cell culture harvest). In some embodiments, a peptide of the present invention has, provides and/or is configured to provide a HCP LRV of at least 2.5. In some embodiments, a peptide of the present invention has, provides and/or is configured to provide a HCP LRV of at least 2.7. For an oligonucleotide and/or polynucleotide (e.g., DNA and/or RNA) from the host organism, a peptide of the present invention may have, provide and/or is configured to provide a LRV of at least about 2 or more (e.g., about 2, 2.5, 3, 3.5, 4, 4.5, or more), optionally wherein the peptide has, provides and/or is configured to provide an oligonucleotide and/or polynuceotide LRV of about 4.

In some embodiments, a peptide of the present invention binds an immunoglobulin (e.g., a polyclonal and/or monoclonal antibody) or fragment thereof. The immunoglobulin may be a polyclonal or monoclonal antibody or a fragment of such an antibody. In some embodiments, the peptide binds the Fc portion of an immunoglobulin or fragment thereof. For example, a peptide of the present invention may bind to the Fc portion of a Fc-fusion protein (e.g., a protein recombinantly expressed as natively connected to the Fc fragment of IgG).

Example immunoglobulins or fragments thereof that a peptide of the present invention may bind include, but are not limited to human IgG (e.g., IgGᵢ, IgG₂, IgG₃, and/or IgG₄), IgA, IgE, IgD, and/or IgM; non-human mammalian (e.g., mouse, rat, rabbit, hamster, horse, donkey, cow, goat, sheep, llama, camel, alpaca, etc.) IgG, IgA, and/or IgM; and/or avian (e.g., chicken, turkey, etc.) IgY.

A peptide of the present invention may comprise a detectable moiety. A “detectable moiety” as used herein refers to any moiety that can be used to detect the peptide including, but not limited to, a fluorescent molecule, a chemiluminescent molecule, a radioisotope, an enzyme substrate, a biotin molecule, an avidin molecule, a chromogenic substrate, an affinity molecule, a protein, a peptide, nucleic acid, a carbohydrate, an antigen, a hapten, and/or an antibody. In some embodiments, the detectable moiety is a portion of the peptide (e.g., an amino acid and/or side chain of an amino acid) and/or the detectable moiety is a moiety that is attached to a portion of the peptide. In some embodiments, a detectable moiety is an antibody, antibody fragment, peptide, nucleic acid sequence, or fluorescent moiety. In some embodiments, a peptide may be photoaffinity labelled, optionally by attaching a photoreactive group, such as a benzophenone group, to the peptide.

Provided according to some embodiments of the present invention is an article comprising a solid support and a peptide of the present invention. In some embodiments, a solid support may comprise a peptide of the present invention, optionally wherein the peptide may be attached (e.g., covalently and/or noncovalently) to a surface of the solid support. In some embodiments, one or more peptide(s) of the present invention, that may be the same or different, may be bound to a solid support (e.g., to a surface of the solid support). In some embodiments, one or more (e.g., 1, 5, 10, 20, 50, 100, 200, 500, or more) copies of the same peptide are bound to a single solid support (e.g., on the surface of the solid support). Example solid supports include, but are not limited to, a chromatographic resin, a membrane, a biosensor, a microbead, a magnetic bead, a paramagnetic particle, a quantum dot, and/or a microplate. In some embodiments, the solid support is a chromatography resin such as a TOYOPEARL® resin. In some embodiments, the solid support is a polymeric resin such as an agarose resin or a methacrylic polymer resin, and optionally the polymeric resin may be configured to bind a peptide (e.g., bind the peptide using a functional group such a hydroxyl group or amine group). In some embodiments, a peptide is covalently bound to a solid support (e.g., to a surface of the solid support). An article of the present invention may be an affinity adsorbent.

An article of the present invention may have density of the peptide in a range of about 0.02, 0.05, 0.1, 0.15, or 0.2 mmol of the peptide per mg of the solid support (mmol/mg) to about 0.25, 0.3, 0.35, 0.4, 0.45, 0.5, 0.55, 0.6, 0.65, 0.7, 0.75, or 0.8 mmol of the peptide per mg of the solid support (mmol/mg). In some embodiments, an article of the present invention includes a peptide of the present invention at a density of about 0.01, 0.02, 0.05, 0.1, 0.15, 0.2, 0.25, 0.3, 0.35, 0.4, 0.45, 0.5, 0.55, 0.6, 0.65, 0.7, 0.75, or 0.8 mmol of the peptide per mg of the solid support (mmol/mg).

In some embodiments, a peptide is attached to a solid support via a covalent linkage. A linking group that may be used to form a covalent linkage may be attached to any portion of the peptide. In some embodiments, a linking group is attached to the N-terminus or C-terminus of a peptide. In some embodiments, a linking group is attached to the C-terminus of a peptide. In some embodiments, the linking group may be selected from —OH, —NH₂, —NHR″, —OR″, —O—NH₂, —O—R″—SH, —O—NH—R″—SH, —O—R″—S—SH, —NH—R″—S—SH, —O—NH—R″—S—SH, an ether, thioether, thioester, carbamate, carbonate, amide, ester, secondary or tertiary amine, or alkyl, wherein R″ is an alkyl. Due to attachment to a solid support, one or more atom(s) (e.g., a hydrogen atom) and/or functional group(s) of the linking group may be removed from the linking group to bind the peptide to the solid support, thereby providing a linking moiety and structure represented by P-Z-R′, wherein P is the peptide, Z is a linking moiety and R′ is a solid support. In some embodiments, Z may be selected from —O—, —NH—, —O—NH—, —O—R″—S—, —O—NH—R″—S—, —O—R″—S—S—, —NH—R″—S—S—, —O—NH—R″—S—S—, ether, thioether, thioester, carbamate, carbonate, amide, ester, amine (e.g., a secondary/tertiary amine optionally obtained through a reductive amination coupling reaction), alkyl (e.g., obtained through a metathesis coupling reaction), alkenyl, phosphodiester, phosphoether, oxime, imine, hydrazone, acetal, hemiacetal, semicarbazone, ketone, ketene, aminal, hemiaminal, enamine, enol, disulphide, sulfone, wherein R″ is alkyl. In some embodiments, a peptide may be attached to a solid support in a manner as described in U.S. 2016/0075734 and/or U.S. Pat. No. 10,266,566.

In some embodiments, an article of the present invention is reusable. An article of the present invention may be used at least 100, 150, or 200 times or more without losing more than about 20% (e.g., about 15%, 10%, 5%, etc.) of its binding capacity after reuse. In some embodiments, an article of the present invention may be sanitized with 0.5 M sodium hydroxide at least 100, 150, or 200 times without losing more than 20% (e.g., 15%, 10%, 5%, etc.) of its binding capacity after sanitization. “Binding capacity” as used herein refers to the amount of target (e.g., immunoglobulin) bound by a given volume of peptide and/or article of the present invention.

According to some embodiments, a method of detecting an immunoglobulin or fragment thereof in a sample is provided, the method may comprise: contacting a sample and a peptide of the present invention under suitable conditions wherein the peptide binds the immunoglobulin or fragment thereof; and detecting the peptide and/or a detectable moiety associated with (e.g., bound to) the peptide, thereby detecting the immunoglobulin or fragment thereof, optionally wherein the peptide is present in the sample or is isolated from the sample. In some embodiments, the peptide is bound to a solid support. In some embodiments, detecting the peptide comprises detecting a detectable moiety that is part of the peptide and/or attached thereto.

In some embodiments, a method of purifying an immunoglobulin or fragment thereof present in a sample is provided, the method comprising: contacting a sample and a peptide of the present invention; and separating (e.g., releasing, eluting, etc.) the immunoglobulin or fragment thereof from the peptide, thereby purifying the immunoglobulin or fragment thereof from the sample. In some embodiments, the peptide is bound to a solid support.

The sample may comprise an immunoglobulin or a fragment thereof, optionally wherein the immunoglobulin or fragment is free in a solution (e.g., an aqueous solution), and may include one or more impurities (e.g., host cell proteins, lipids, etc.). In some embodiments, the sample is and/or is obtained from a cell culture fluid (e.g., supernatant), a plant extract, a bodily fluid (e.g., human blood and/or plasma, transgenic milk, etc.), and/or a feedstock (e.g., a cellular feedstock). A cell culture fluid may comprise a plurality of cells such as, but not limited to, mammalian cells, (e.g., Chinese hamster ovary (CHO) cells, human embryonic kidney (HEK) 293 cells), bacterial cells, and/or yeast cells (e.g., Pichia pastoris cells).

The contacting step in a method of the present invention may be carried out under suitable conditions such that a target immunoglobulin or fragment thereof is bound to and/or immobilized with the peptide. The contacting step is carried out to bring the peptide and target together or in sufficient proximity such that, under suitable conditions, the target is bound to and/or immobilized with the peptide. The target immunoglobulin or fragment may be bound to the peptide covalently and/or non-covalently. In some embodiments, the target immunoglobulin or fragment may be bound to the peptide via affinity adsorption. During the contacting step, the target immunoglobulin or fragment may bind to the peptide and the impurities (e.g., HCPs) in the sample may not bind to the peptide. In some embodiments, a sample is contacted to a plurality of articles of the present invention (e.g., solid supports comprising a peptide of the present invention) and one or more impurities do not bind to the peptide and/or flow through the plurality of articles, thereby at least partially separating the target (e.g., immunoglobulin or fragment) from the impurities (e.g., HCPs).

In some embodiments, a method of the present invention comprises washing an article of the present invention following target (e.g., immunoglobulin) binding, which may remove one or more impurities. In some embodiments, washing the article removes one or more impurities that are non-specifically adsorbed onto the article and/or peptide. Washing may be performed prior to separating (e.g., releasing) an immunoglobulin or fragment from a peptide and/or article.

A method of the present invention may comprise separating (e.g., releasing, eluting, etc.) an immunoglobulin or fragment from a peptide and/or article thereby providing an isolated immunoglobulin or fragment. Separating or releasing the immunoglobulin or fragment from the peptide and/or article may comprise an elution step. In some embodiments, separating or releasing the immunoglobulin or fragment from the peptide and/or article comprises eluting the immunoglobulin or fragment from the peptide and/or article. Eluting the immunoglobulin or fragment from the peptide and/or article may comprise contacting an aqueous buffer that is suitable to disrupt the peptide-immunoglobulin interaction such that the immunoglobulin or fragment is separated or released from the peptide. The aqueous buffer suitable to disrupt the peptide-immunoglobulin interaction may comprise a compound (e.g., a salt) in a concentration sufficient to disrupt the interaction and/or a have a pH sufficient to disrupt the interaction.

In some embodiments, a method of the present invention may comprise one or more affinity chromatography steps, either in series or parallel, which may be used to isolate and/or purify an immunoglobulin or fragment thereof.

A method of the present invention may further comprise determining the amount and/or purity of an isolated immunoglobulin or fragment after a separating step. An HCP-specific ELISA may be used to determine the amount of HCPs in a composition (e.g., an eluted fraction) comprising the isolated immunoglobulin or fragment. Comparison of the concentration of HCPs in the composition compared to the amount of HCPs in the initial sample may be used to determine the amount and/or purity of the isolated immunoglobulin or fragment, optionally to provide a HCP LRV for the isolated immunoglobulin or fragment. In some embodiments, a method of the present invention provides a composition comprising the isolated immunoglobulin or fragment and the composition may have a HCP concentration in a range of about 0, 0.25, 0.5, 0.75, 1, 1.5, or 2 mg of HCP per mL of the composition to about 2.5, 3, 3.5, 4, 4.5, or 5 mg of HCP per mL of the composition. In some embodiments, a method of the present invention provides a composition comprising the isolated immunoglobulin or fragment and the composition may have a HCP concentration of about 0, 0.5, 0.75, 1, 1.5, 2, 2.5, 3, 3.5, 4, 4.5, or 5 mg of HCP per mL of the composition.

A method of the present invention may provide a purity of the isolated immunoglobulin or fragment thereof of at least 80% after a separating step. In some embodiments, the purity of the isolated immunoglobulin or fragment thereof, after a separating step, is at least about 80%, 81%, 82%, 83%, 84%, 85%, 86%, 8′7%, 88%, 89%, 90%, 91%, 92%, 93%, 94%, 95%, 96%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, or 100% or any value or range therein. In some embodiments, the purity of the isolated immunoglobulin or fragment thereof, after a separating step, is at least about 97% and the LRV is at least about 2.5. In some embodiments, the purity of the immunoglobulin or fragment thereof, after a separating step, is at least about 98.1% and the LRV is at least about 2.7. The peptides of the present invention may be used to bind to, collect, purify, immobilize on a solid surface, etc., any type of antibody or Fc-fragment comprising compound (e.g., Fc-fusion proteins), including both natural and recombinant (including chimeric) antibodies, engineered multibodies, and combinations thereof, such as divalent antibodies and camelid immunoglobulins, and both monoclonal and polyclonal antibodies, or an Fc-fusion protein. The antibodies may be of any species of origin, including mammalian (rabbit, mouse, rat, cow, goat, sheep, llama, camel, alpaca, etc.), avian (e.g., chicken, turkey, etc.), shark, etc., including fragments, chimeras and combinations thereof as noted above. The antibodies may be of any type of immunoglobulin, including but not limited to IgG, IgA, IgE, IgD, IgM, IgY (avian), etc.

In some embodiments, the antibodies or Fc fragments (including fusion proteins thereof) are carried in a biological fluid such as blood or a blood fraction (e.g., blood sera, blood plasma), egg yolk and/or albumin, tissue or cell growth media, a tissue lysate or homogenate, etc.

According to some embodiments, provided is a method of binding an antibody or antibody Fc fragment from a liquid composition (e.g., a sample) containing the same, the method comprising providing an article comprising a solid support and a peptide of the present invention; contacting said composition to said article so that the antibody or Fc fragment or Fc-fusion protein bind to said peptide; and separating said liquid composition from said article, with said antibody or Fc fragment or Fc-fusion protein bound to said article; optionally washing (but in some embodiments preferably) said article to remove HCPs non-specifically bound to the article; and optionally (but in some embodiments preferably) separating (e.g., eluting) said antibody or Fc fragment or Fc-fusion protein from said article, thereby providing the antibody or antibody Fc fragment in an isolated and/or purified form.

A method of the present invention may be carried out in like manner to those employing protein A, or by variations thereof that will be apparent to those skilled in the art. For example, the contacting and separating steps can be carried out continuously, (e.g., by column chromatography), after which the separating step can then be carried out (e.g., by elution), in accordance with known techniques. In some embodiments, a method of the present invention comprises one or more steps as described in U.S. 2016/0075734 and/or U.S. Pat. No. 10,266,566.

In some embodiments, when the liquid composition and/or sample from which the immunoglobulin or fragment thereof (e.g., antibodies or Fc fragments or Fc-fusion proteins) is to be collected comprises a biological fluid, the liquid composition may further comprise at least one proteolytic enzyme. In some embodiments, a peptide of the present invention is resistant to degradation by proteolytic enzymes.

The following examples are provided solely to illustrate certain aspects of the particles and compositions that are provided herein and thus should not be construed to limit the scope of the claimed invention.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Identification of Novel Peptide Protein A Mimetics for mAb Purification.

Synthetically manufactured peptides have been investigated as specifically-binding biorecognition moieties for diagnostics (Liu et al. 2015 Talanta 136:114-127; Pavan and Berti 2012 Analytical and Bioanalytical Chemistry 402:3055-3070; Hussain et al. 2013 Biosensors 3:89-107), therapeutics (Fosgerau and Hoffman 2015 Drug Discovery Today 20(1):122-128), and protein purification (Menegatti et al. 2013 Pharmaceutical Bioprocessing 1(5):467-485). Numerous peptide ligands have been developed during the last two decades targeting a wide variety of protein therapeutics, including human antibodies, blood proteins, hormones, and enzymes. Binding capacity values, product recovery, and purity obtained with peptide-based adsorbents demonstrate that peptides are a credible alternative to protein ligands. The IgG-binding peptide ligand HWRGWV (SEQ ID NO:18) has been extensively characterized (Yang et al. 2006 J. of Peptide Research 66:120-137; Yang et al. 2009 J. of Chromatography A 1216(6):910-918). This ligand, which has an optimized HCP LRV of 1.6 (Naik et al. 2011 J. of Chromatography A 1218:1691-1700), has been shown effective at recovering monoclonal and polyclonal antibodies from a variety of complex sources, including cell culture fluids (Naik et al. 2011), plant extracts (Naik et al. 2012 J. of Chromatography A 1260:61-66), human plasma (Liu et al. 2012 J. of Chromatography A 1262:169-179; Menegatti et al. 2012 J. of Separation Science 35:3139-3148; Menegatti et al. 2016 1 of Chromatography A 1445:93-104), and transgenic milk (Menegatti et al. 2012). In recent work on the optimization of HWRGWV (SEQ ID NO:18)-based adsorbents, resins with binding capacity of up to 91.5 mg of IgG per mL of adsorbent (Menegatti et al. 2016). Variants of HWRGWV (SEQ ID NO:18) have also been developed using non-natural amino acids to ensure resistance against proteolytic enzymes. Notably, the variant Ac-HWCitGWV (Ac-: acetylated N terminus, Cit: citrulline; SEQ ID NO:20), upon optimized binding and washing conditions, offered a HCP LRV of 2.07. This indicates that optimizing the amino acid composition and sequence of HWRGWV (SEQ ID NO:18) can lead to new ligands with significantly higher binding selectivity.

In this study, a peptide search algorithm developed and validated in prior work (Xiao et al. 2015 J. of Chemical Theory and Computation 11:740-752; Xiao et al. 2018 ACS Sensors 3:1024-1031; Xiao et al. 2017 J. of Chemical Theory and Computation 13(11):5709-5720; Xiao et al. 2015 J. of Biomolecular Structure an Dynamics 33(1):14-27; Xiao et al. 2016 J. of Computational Chemistry 37(27): 2433 -2435; Xiao et al. 2016 Proteins: Structure, Function and Bioinformatics 84(5):700-711) was used to design sequence variants of HWRGWV (SEQ ID NO:18) with higher binding selectivity to IgG. Initially, the structure of the IgG-HWRGWV (SEQ ID NO:18) complex was analyzed to identify the topological and physicochemical properties of its binding site. Thereafter, the Autodock program was used to locate alternative, more-likely binding sites. The peptide design algorithm was then used to screen 60,000 sequence variants of HWRGWV (SEQ ID NO:18) on the alternative IgG binding site. Sequence variation was constrained to fix the peptide charge (−1 to +3) and the hydrophobicity (a maximum of 2 aromatic amino acids) based on knowledge of the IgG-HWRGWV (SEQ ID NO:18) complex. The variants were ranked according to a “Γ score”, which measures each variant's binding internal energy (electrostatic, van der Waals, solvation, etc.) to the target and its stability in the bound conformation. The Monte Carlo (MC) Metropolis algorithm was used to accept or reject the new peptide sequence, thereby evolving the peptide sequence to those with the best Γ scores. Finally, the binding energies of the 10 peptide variants with the highest Γ score were evaluated by running at least three independent explicit-solvent atomistic molecular dynamics (MD) simulations of each peptide-protein complex. The MD simulations start from the configuration returned by the search algorithm and enable peptide and protein flexibility, allowing them to evolve to their equilibrium configurations. The search algorithm returned four variants, WQRHGI (SEQ ID NO:1), MWRGWQ (SEQ ID NO:2), RHLGWF (SEQ ID NO:3), and GWLHQR (SEQ ID NO:4), which had low predicted binding energies. A second set of studies was conducted in which the four sequences were screened in silico against a panel of 14 HCPs via molecular docking to ensure that the chosen ligands were selective. The combined results of MD simulations and docking to HCPs were confirmed in vitro, showing RHLGWF (SEQ ID NO:3) to be non-selective and GWLHQR (SEQ ID NO:4) to have lower than expected IgG yields.

Sequences WQRHGI (SEQ ID NO:1) and MWRGWQ (SEQ ID NO:2), which had the best performance in computational and initial competitive binding studies, were chosen for further experimental evaluation. These ligands were conjugated on agarose-based WorkBeads resins and then evaluated experimentally in terms of their static binding strength and capacity (K_{D(Solid)} and Qₘₐₓ), dynamic binding capacity (DBC_{10%}), and ability to purify IgG from a CHO cell culture fluid. The WQRHGI (SEQ ID NO:1)—WorkBeads resins and MWRGWQ (SEQ ID NO:2)—WorkBeads resins showed values of K_{D(Solid)} (3.2×10⁻⁶ M and 8.14×10⁻⁶, respectively), Qₘₐₓ (52.6 and 57.5 mg/mL) and DBC_{10%} (43.8 and 55.3 mg/mL, at 5 min residence time) which were similar to corresponding values measured on HWRGWV (SEQ ID NO:18)—Workbeads resin in prior work. Yet, the WQRHGI (SEQ ID NO:1)—WorkBeads afforded a remarkably higher value of HCP LRV, 2.7, with minimal optimization of the chromatographic protocol. To further corroborate the in silico design, an ensemble of variants of WQRHGI (SEQ ID NO:1) and MWRGWQ (SEQ ID NO:2) were constructed by replacing residues indicated by the algorithm as key binders with amino acids carrying different functionalities. Almost all of the resulting sequence variants showed poor IgG binding, thereby supporting the in silico decomposition of energy of binding by amino acid. Collectively, these results portray the peptide WQRHGI (SEQ ID NO:1) as a valid alternative to Protein A for the capture step in a platform purification process for mAb therapeutics.

Sodium chloride, glycine, iodoacetic acid (IAA), 1-ethyl-3-(3-dimethyl aminopropyl) carbodiimide hydrochloride (EDC), N,N′-dimethylformamide (DMF), bicinchoninic acid (BCA) protein concentration assay, and Silver Quest Silver Stain kit were purchased from Fisher (Pittsburgh, PA). 4-20% Bis-Tris Mini-PROTEAN gels were purchased from BioRad, run on a Bio-Rad TetraCell with Precision Protein Plus Dual Color protein standard, and stained using BioRad Bio Safe coomassie (Hercules, CA) or aforementioned Silver Quest silver stain kit. Potassium chloride, potassium phosphate monobasic, phosphate buffered saline (PBS) at pH 7.4, (β-mercaptoethanol, triethylamine, ethanedithiol, anisole, and thioanisole were from Sigma Aldrich (St. Louis, Missouri).

Triuoroacetic acid (TFA), Fmoc-protected amino acids, piperidine, diisopropylethylamine (DIPEA), and Hexauorophosphate Azabenzotriazole Tetramethyl Uronium (HATU) were purchased from Chem Impex (Wood Dale, Illinois). Sodium phosphate di-basic and methanol were purchased from VWR/Amresco (Solon, Ohio). Chromatographic experiments were performed on a Waters 2695 separations platform. Microbore PEEK columns 30 mm long 2.1 mm I.D. were purchased from VICI Precision Sampling (Baton Rouge, Louisiana, USA). IgG was purchased from Athens Research & Technology (Athens, Georgia, USA). Chinese hamster ovary (CHO) cell culture supernatant was generously provided by the Biomanufacturing Training and Education Center (BTEC) at NC State University. The CHO HCP ELISA assays were purchased from Cygnus Technologies (Southport, NC). Workbeads 40 TREN resins were purchased from BioWorks (Uppsala, Sweden). Purified peptide ligands were synthesized by Genscript (Piscataway, NJ).

Peptide design algorithm: The peptide design algorithm used in this study was previously proven capable of discovering peptide sequences with higher binding strength than a known “reference ligand”, and was used in this study to produce variants of the reference peptide HWRGWV (SEQ ID NO:18) that bind human IgG with higher affinity. The complex of HWRGWV (SEQ ID NO:18) with the Fc region of human IgG was utilized as a reference in docking studies to identify a new initial binding site for the peptide on IgG. Sequence evolution was conducted on peptides in the form X₁X₂X₃X₄X₅X₆GSG to generate 6-mer IgG-binding peptide sequences. The GSG (Gly-Ser-Gly) trimer on the peptide C-terminal was added as a non-binding segment to simulate the orientation that the peptide ligand assumes when conjugated onto the chromatographic support. This trimer was stipulated to be non-interacting during binding simulations. During sequence variations either one randomly chosen amino acid was mutated or two randomly chosen amino acids on the peptide were exchanged. The numbers of positively-charged, negatively-charged, hydrophobic, polar, or other residues chosen during sequence moves were constrained to fine tune the biochemical function of the peptide variants. There were two types of trial “moves” in the computational algorithm: peptide sequence change moves during which the peptide conformation within the complex was fixed, and peptide conformation change moves during which the peptide sequence was fixed. The target molecule's conformation was fixed. The side-chain conformations of the amino acids were taken from Lovell's rotamer library, and each resulting variant was subjected to energy minimization to determine the optimal configuration. A “Γ score” that measures each variant's binding internal energy (van der Waals, electrostatic, solvation, etc.) to the target and its stability in the bound conformation was then evaluated using implicit-solvent MM/GBSA approach with the AMBER14SB force field. The Monte Carlo Metropolis algorithm was used to accept or reject the new peptide variant, thereby evolving the peptide sequence to those with the lowest Γ scores. At the end of 10,000 iterations, the peptide variants with the lowest scores were identified. The binding free energies of selected peptide variants (those with the lowest Γ scores) for target molecule IgG were evaluated by three independent runs of 100-ns explicit-solvent atomistic MD simulations on each peptide-protein complex. The MD simulations start from the configuration returned by the search algorithm and enable peptide and protein flexibility, allowing them to evolve to their equilibrium configurations.

Docking of peptides WQRHGI (SEQ ID NO:1) and MWRGWQ (SEQ ID NO:2) on model HCPs: Putative binding sites on a selection of HCPs were found using a druggability assessment to identify likely binding sites. Herein, protein “druggability” was determined using PockDrug. These studies indicate those surfaces and pockets most likely to be targeted by small moleculeor peptide ligand.

The selected HCPs and the number of potential binding sites for each HCP investigated are delineated in Table 2. The PDB IDs of the crystal structures used in this study are presented in the table; unfortunately, the crystal files of the listed “problematic” HCPs from Chinese hamster (Cricetulus griseus) are not available on the Protein Data Bank. In order to use the most homologically similar proteins, the murine (Mus musculus) and rat (Rattus norvegicus) forms of the proteins were utilized when available. When the protein structures were not available for rodents, the human forms were utilized or, barring that, drosophila (Drosophila melanogaster). It was stipulated that these proteins are homologous to the Chinese hamster proteins and can serve in this capacity as a negative screening tool. The number of putative binding sites on each HCP are listed in the final column of the table.

TABLE 2
HCPs used in study

| Protein | Organism | PDB ID | Sites |
|---|---|---|---|
| Carboxypeptidase A | Human | 5OM9 | 4 |
| Carboxypeptidase D | Drosophila | 3MN8 | 3 |
| Cathepsin D | Human | 4OD9 | 2 |
| Cathepsin D | Murine | 5UX4 | 1 |
| Cathepsin L | Human | 5MAE | 1 |
| Enolase 1 | Human | 2PSN | 4 |
| Enolase 1 | Human | 5MBL | 3 |
| Enolase 1 | Human | 1THE | 2 |
| Glutathione S-transferase | Human | 5J41 | 3 |
| Glutathione S-transferase | Murine | 3O76 | 3 |
| Lipoprotein lipase | Human | 6E7K | 3 |
| Peroxiredoxin | Human | 3HY2 | 2 |
| Peroxiredoxin 1 | Rat | 2Z9S | 3 |
| Peroxiredoxin 4 | Murine | 3VWU | 2 |

Peptides WQRHGI (SEQ ID NO:1), MWRGWQ (SEQ ID NO:2), RHLGWF (SEQ ID NO:3), and GWLHQR (SEQ ID NO:4) were docked in silico against the putative binding sites on the crystal structures of the Table 2 listed HCPs using the docking software HADDOCK (High Ambiguity Driven Protein-Protein Docking, v.2.1). The resulting HCP:peptide dockings were individually clustered based on a fraction of common contacts, wherein a “cluster” was defined as a collection of at least four structures with 85% similar contacts or better. The binding energy of the selected HCP:peptide complexes within the most highly populated clusters was determined using the PRODIGY (PROtein binDIng enerGY prediction) webserver. The resulting configurations between peptides and HCPs were then simulated using AMBER15 with an explicit solvent approach to examine the kinetic process of the binding of peptide variants to each of the 14 HCPs.

Peptide synthesis: Sequences WQRHGI (SEQ ID NO:1), MWRGWQ (SEQ ID NO:2), RHLGWF (SEQ ID NO:3), and GWLHQR (SEQ ID NO:4) derived from the in silico ligand search, and variants MFRGWQ (SEQ ID NO:21), MWRAWQ (SEQ ID NO:5), MWRGFQ (SEQ ID NO:7), MWRGWN (SEQ ID NO:22), (NorL)WRGWQ (NorL: nor-leucine; SEQ ID NO:23), MGRGWQ (SEQ ID NO:24), MW(Cit)GWQ (Cit: citrulline; SEQ ID NO:25), MWRWQ (SEQ ID NO:6), MWRGGQ (SEQ ID NO:26), GWRGWQ (SEQ ID NO:8), WQRHGIC (SEQ ID NO:30), WNRHGI (SEQ ID NO:12), WQ(Cit)HGI (SEQ ID NO:27), WQRAGI (SEQ ID NO:28), WQRHAI (SEQ ID NO:11), WQRHGL (SEQ ID

NO:9), FQRHGI (SEQ ID NO:29), and WQRHGV (SEQ ID NO:10) were synthesized on Toyopearl AF-Amino 650 M chromatographic resin (amino functional density: 0.6 mmol/mL, Tosoh, Tokyo, Japan) using a Biotage Syro I robotic liquid handler and peptide synthesis suite (Biotage, Charlotte, NC) following the Fmoc/tBu strategy. Every residue was conjugated using three couplings with Fmoc-protected amino acid (2.4-fold molar excess compared to the amino functional density on Toyopearl resin), HATU (2.8-fold molar excess), and DIPEA (3-fold molar excess) in dry DMF, at 75° C. for 12 minutes. Fmoc deprotection was performed using 40% piperidine in DMF for 4 minutes, followed by 20% piperidine in DMF for 15 minutes at room temperature. Final peptide deprotection was performed by acidolysis for 2 hours, using a cocktail of 90:5:3:2 TFA:thioanisole:ethanedithiol:anisole. The resins were finally dried in dichloromethane and stored at −20° C. until swollen in 20% methanol.

Peptide conjugation on WorkBeads TREN resins: Aliquots of 5mL of WorkBeads TREN resins were activated using 1.86 g of IAA, 1.55 g of EDC, and 1.12 g NHS as a coupling agent in 12.75 mL of 100 mM IVIES buffer, pH 4.5. The reaction was conducted at room temperature for 48 hours under rotation. To test for completion of this reaction, 10 μL of resin was incubated with an excess of ethane dithiol. The presence of free sulfhydryl groups was then tested using an Ellman assay; 67% of the resin's surface amines were iodo-activated. MWRGWQ (SEQ ID NO:2) was conjugated by incubating 101 mg of peptide at 50 mg/mL in 5% v/v TEA in DMF with 0.4 mL activated resin at room temperature, for 48 hours, in dark, under mild stirring. WQRHGIC (SEQ ID NO:30) was conjugated by incubating 103 mg of peptide at 50 mg/mL in 100 mM phosphate buffer added with 5 mM EDTA at pH 8, with 0.4 mL activated resin at room temperature, for 48 hours, in dark, under mild stirring. The unreacted iodoacetyl groups were saturated using a 5x-excess of 2-mercaptoethanol (50 μL) in 2 mL of DMF containing 10% (v/v) of TEA. The resin was rinsed and stored in 20% v/v ethanol at 4° C. Unreacted iodoacetyl groups on the resin were saturated using 2-mercaptoethanol in 5% v/v TEA in DMF. The unconjugated peptides in solution were quantified by UV absorbance at 280 nm, and the ligand density on the resin was determined via mass balance. The MWRGWQ (SEQ ID NO:2)—Workbeads had a peptide density of 0.43 mmol/mL, while WQRHGIC (SEQ ID NO:30)—Workbeads had a peptide density of 0.110 mmol/mL. The resins were stored at 4° C. in 20% methanol until further use.

Measurement of IgG binding by peptide-based chromatographic adsorbents: For initial studies, 35 mg of MWRGWQ (SEQ ID NO:2)-Toyopearl, RHLGWF (SEQ ID NO:3)-Toyopearl, WQRHGI (SEQ ID NO:1)-Toyopearl, GWLHQR (SEQ ID NO:4)-Toyopearl, and HWRGWV (SEQ ID NO:18)-Toyopearl (control) resins were equilibrated in PBS pH 7.4, reaching a swollen volume of 0.1 mL, and subsequently incubated with 1 mg/mL IgG in 0.205 mg/mL CHO cell culture supernatant for 30 minutes. The resins were subsequently washed several times with PBS to remove non-specifically bound proteins. Elution was performed with 100 mM glycine buffer pH 2.5. Flowthrough and elution fractions were collected and analyzed by SDS PAGE under reducing conditions. The resulting gels were stained with Coomassie staining. Further, 25 mg of the adsorbents MWRGWQ (SEQ ID NO:2)-Toyopearl, NIFRGWQ (SEQ ID NO:21)-Toyopearl, MWRAWQ (SEQ ID NO:5)-Toyopearl, MWRGFQ (SEQ ID NO:7)-Toyopearl, MWRGWN (SEQ ID NO:22)-Toyopearl, (NorL) WRGWQ (SEQ ID NO:23)-Toyopearl, MGRGWQ (SEQ ID NO:24)-Toyopearl, MWRWQ (SEQ ID NO:6)-Toyopearl, MWRGGQ (SEQ ID NO:26)-Toyopearl, GWRGWQ (SEQ ID NO:8)-Toyopearl, WQRHGI (SEQ ID NO:1)-Toyopearl, WNRHGI (SEQ ID NO:12)-Toyopearl, WQRAGI (SEQ ID NO:28)-Toyopearl, WQRHAI (SEQ ID NO:11)-Toyopearl, WQRHGL (SEQ ID NO:9)-Toyopearl, FQRHGI (SEQ ID NO:29)-Toyopearl, and WQRHGV (SEQ ID NO:10)-Toyopearl resins were equilibrated in PBS pH 7.4, reaching a swollen volume of 0.1 mL, and subsequently incubated with 1 mg/mL IgG in PBS at pH 7.4 for 30 minutes. The amount of unbound IgG in the supernatant samples was quantified by Bradford assay and utilized to determine the IgG binding % by the peptide variants.

Measurements of static and dynamic binding capacity: MWRGWQ (SEQ ID NO:2)—Workbeads and WQRHGIC (SEQ ID NO:30)—Workbeads were characterized in terms of static and dynamic binding capacity respectively by batch and breakthrough binding studies. The peptides RHLGWF (SEQ ID NO:3) and GWLHQR (SEQ ID NO:4) were not selected for further studies due to their low selectivity and low yield, respectively. Aliquots of 30 μL of resin were individually incubated with gentle rotation overnight at 4° C. in 200 μL of solution of human polyclonal IgG in PBS at pH 7.4 at different concentrations, namely 0.5, 2, 4, 6, 8, and 10 mg/mL. The resin was pelleted by centrifugation and the supernatant removed. The resins were then washed twice with 100 μL of PBS, and the supernatants were collected. The resulting fractions were combined and analyzed by BCA assay to quantify the unbound IgG and, accordingly, the amount of IgG adsorbed. The resulting data were fit to a Langmuir isotherm to determine the values of Qₘₐₓ and K_{D(Solid)}.

Measurements of dynamic binding capacity (DBC) were performed on a Waters 2695 unit. MWRGWQ (SEQ ID NO:2)—Workbeads and WQRHGIC (SEQ ID NO:30)—Workbeads resins were wet packed in a 0.1 mL microbore column and equilibrated in PBS pH 7.4. A solution of human IgG at 20 mg/mL in PBS was owed through the column at 0.05 mL/min and 0.02 mL/min, corresponding to residence times (RT) of 2 and 5 min, respectively. The bound IgG was eluted with glycine pH 2.5. The absorbance of the effluent was monitored by UV/Vis spectrophotometry at 280 nm throughout the breakthrough study. The DBC was calculated at 10% of the breakthrough curve.

Measurements of IgG-binding affinity in solution by isothermal titration calorimetry (ITC): Experimental determination of the binding free energy of the IgG:WQRHGI (SEQ ID NO:1) complex was performed by ITC using a Nano ITC Low Volume calorimeter (TA Instruments, New Castle, DE). All titration experiments for determining binding enthalpy and affinity were conducted at 25° C. by performing repeated injections (250 sec intervals) of 5 μL of a 2mg/mL solution of WQRHGI (SEQ ID NO:1) in PBS, pH 7.4, into 300 mL of 5 mg/mL solution of polyclonal IgG in PBS, pH 7.4. All solutions were filtered through a 0.22 p.m syringe filter prior to use. Ten injections were performed for each measurement. Background energy from peptide dilution was determined by performing 10 injections of 5 μL of a 2 mg/mL solution of WQRHGI (SEQ ID NO:1) in PBS pH 7.4. The titration data were analyzed using NanoAnalyze software (TA Instruments) and plotted using an independent fitting, which fits the resultant Wiseman plot with parameters corresponding to a non-competitive single-site binding phenomenon in order to calculate the binding affinity (K_{(D(ITC)}), and the stoichiometry (N) of the interaction. A constant blank was also utilized in the fitting to account for the heat of dilution of the IgG substrate.

MWRGWQ (SEQ ID NO:2) was unable to be examined via ITC. Peptide MWRGWQ (SEQ ID NO:2) was not soluble in pH 7.4 buffer, likely due to self-associative properties. MWRGWQ (SEQ ID NO:2) was found soluble in highly acidic buffer, but ITC results were confounded by the heat of mixing between acidic and neutral solutions. Binding of the peptide was also significantly reduced at lower pH, further complicating results. Attempts were made to raise the pH of buffer in which MWRGWQ (SEQ ID NO:2) was dissolved, but the peptide was seen to gel when the pH was raised above 5.

Purification of IgG from CHO Cell culture fluids using MWRGWQC (SEQ ID NO:31)- and WQRHGIC (SEQ ID NO:30)—Workbeads: A volume of 0.1 mL of resin was packed in a PEEK microbore column, installed on a Waters 2695 unit, and equilibrated with PBS, pH 7.4. All chromatographic buffers were filtered through a compatible 0.2 μm filter prior to use. A volume of 100 μL of solution of human polyclonal IgG at 1 mg/mL in a CHO cell culture fluid at 0.205 mg/mL CHO HCPs was injected in the column at 0.02 mL/min (RT: 5 minutes). Following injection, the resin was washed with PBS at 0.2 mL/min and, subsequently, with 100 mM NaCl in PBS at 0.2 mL/min. Elution was then conducted with M acetate buffer pH 4. An acidic cleaning step was conducted in 0.1 M glycine pH 2.5 to remove any proteins still bound. The absorbance of the effluent was monitored by UV/Vis spectrophotometry at 280 nm. Fractions were collected and adjusted to neutral pH. Total protein concentration was measured by BCA assay. All collected fractions were also analyzed via SDS PAGE under reducing conditions. The gel was stained by silver staining, and the overall IgG purity in the eluted fractions was determined by densitometric analysis using ImageJ software. Finally, the feed and eluted fractions were analyzed using a CHO-specific ELISA kit to determine the log removal value (LRV) of HCPs.

In silico search for peptide binders: Using the methods described above, a large number of sequences were generated and investigated. The amino acids chosen for mutation moves were completely un-biased during the first round of in silico screening. In the second and subsequent rounds, the mutations were restricted to have at most only one of the following amino acids in the sequence: Leu, Val, Ile, Ala, Trp, His, Arg, Lys, Ser, Thr, Asn, Gln, and Gly. This was done to limit the number of hydrophobic amino acids (Leu, Val, Ile, Ala, Trp) and thus reduce non-specific hydrophobic interactions. Positively charged amion acids (His, Arg, Lys) can contribute to non-specific electrostatic and ionic interactions and were limited to prevent discovery of ion-exchange-like ligands.

Because previously published designs had purported binding sites on CH3, initial studies and peptide designs were conducted using a binding site on the CH3 portion of IgG. However, due to the natural overlap of CH3 subunits at the area where designs showed highest likelihood of binding, alternative sites were later sought. Since IgG chains CH2 and CH3 have high levels of homology and extremely similar residue qualities (alignment of RMSD: 3.16 Å and similarity: 39/113, or 34.5%), CH2 was considered a reasonable target for IgG binding. To this end, the peptides discovered using the CH3 portion were then docked and atomistically simulated, but on the CH2 fragment instead of CH3. These simulations were carried out in explicit-solvent model for 100 ns, the last 10 ns of which were used for pose analysis and the free energies of the four ligand candidates were then calculated using the implicit-solvent MM/GBSA approach with the variable internal dielectric constant model.

TABLE 3
Scores for candidate peptide sequences

| Sequence | Γ Score | ΔG_{b(MD)} (kcal/mol) |
|---|---|---|
| HWRGWV | −22.61 | −8.19 |
| (SEQ ID NO: 18) | | |
| WQRHGI | −21.72 | −8.81 |
| (SEQ ID NO: 1) | | |
| MWRGWQ | −34.2 | −8.59 |
| (SEQ ID NO: 2) | | |
| RHLGWF | −30.55 | −8.43 |
| (SEQ ID NO: 3 | | |
| GWLHQR | −35.17 | −15.17 |
| (SEQ ID NO: 4) | | |

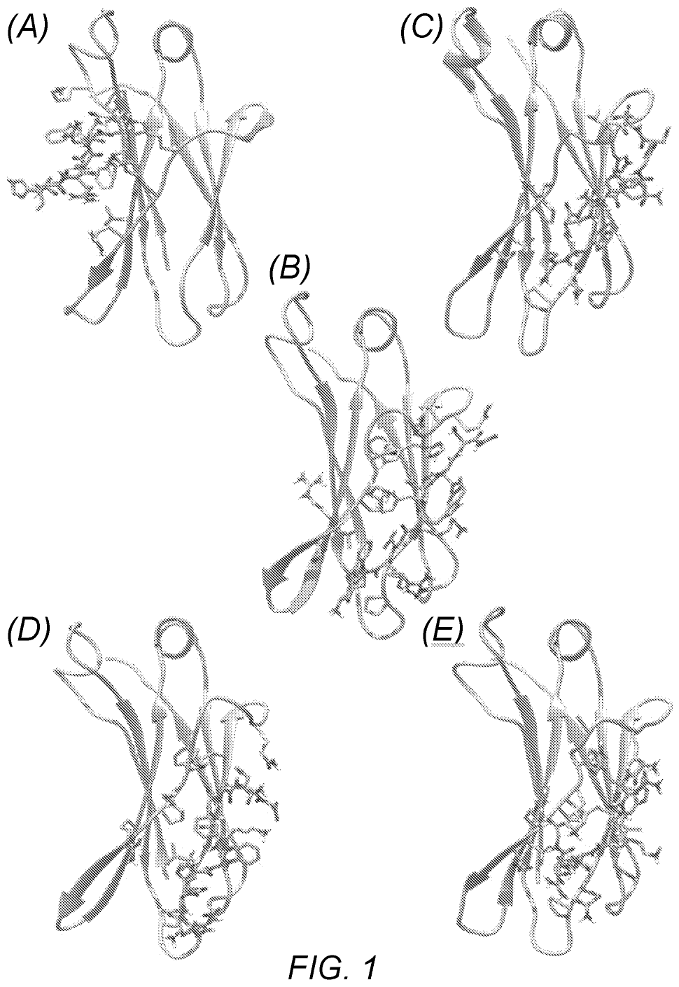

Among the identified sequences, four candidates were selected for further evaluation, namely WQRHGI (SEQ ID NO:1), MWRGWQ (SEQ ID NO:2), RHLGWF (SEQ ID NO:3), and GWLHQR (SEQ ID NO:4), which were shown to have a computed binding free energy ΔG_{b(MD)} of −8.81 kcal/mol, −8.59 kcal/mol, −8.43 kcal/mol, and −15.17 kcal/mol, respectively. All of these binding energies were lower than HWRGWV's (SEQ ID NO:18) −8.19 kcal/mol, as detailed in Table 3. The values of ΔG_{b(MD)} still have notable deviations from experimentally-measured values; for instance, ΔG_{b(MD)}=−15.17 kcal/mol for GWLHQR (SEQ ID NO:4). One reason for this is that the MM/GBSA approach used for the post-analysis of the simulation trajectories neglects the effect of water, and hence does not give estimates of the enthalpy and entropy contributed by solvation. When binding events occur, they are accompanied by the dissociation of water from the peptides and from IgG. This results in an increase in the freedom of motion for water, thereby causing a loss of enthalpy and a gain of entropy. Nevertheless, WQRHGI (SEQ ID NO:1), RHLGWF (SEQ ID NO:3), and GWLHQR (SEQ ID NO:4) were chosen for in vitro investigation because of their low Γ scores and low values of ΔG_{b(MD)} derived from the explicit solvent atomistic MD simulations. MWRGWQ (SEQ ID NO:2) resembles the reference sequence HWRGWV (SEQ ID NO:18), and was thus also selected for further experimental evaluation. The replacement of His with Met in position 1 was of particular interest. In the original work on the discovery of HWRGWV (SEQ ID NO:18), in fact, a preponderant presence of His in position 1 (peptide N terminus) was highlighted as one of the main sequence homology features among the sequences identified from library screening. The complexes formed by sequences WQRHGI (SEQ ID NO:1), MWRGWQ (SEQ ID NO:2), RHLGWF (SEQ ID NO:3), and GWLHQR (SEQ ID NO:4) with the CH2 region of human IgG (PDB ID:1FCC) are reported in FIG. 1.

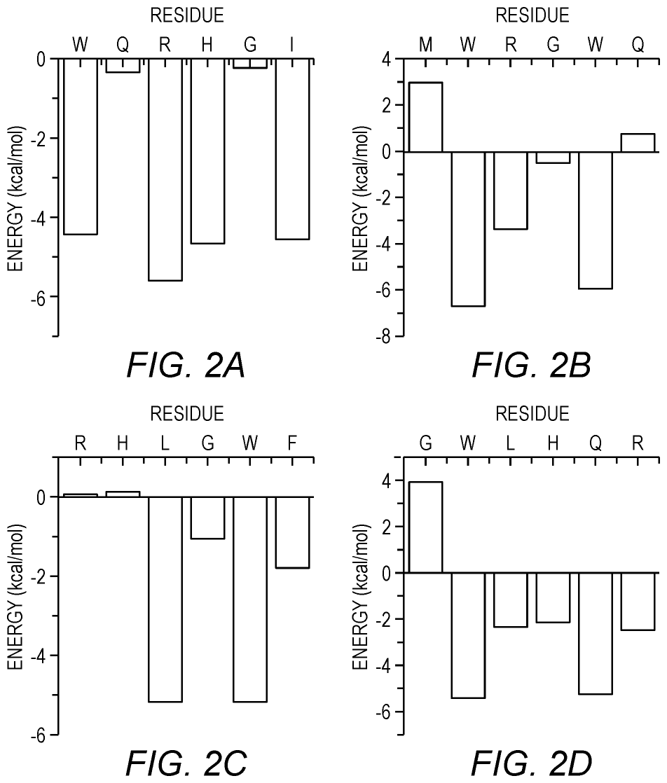

The individual residue contributions to the binding energy were also calculated using explicit solvent simulations with post analysis via the MM/GBSA approach as graphically shown in FIG. 2. This information offers insight regarding the driving forces governing the IgG-peptide binding and dissociation. It also shows the relative importance of the different residue characteristics such as hydrophobicity, charge, or structure, and was used to inform our choices of a select library of sequence variants for in vitro study.

In silico evaluation of peptide selectivity: When utilized as affinity ligands for the purification of mAbs from recombinant sources, the peptides must be able to recognize the target IgG molecules in a complex environment comprising hundreds of secreted HCPs.

Current literature on the secretome of Chinese Hamster Ovary (CHO) cells, the established workhorse in industrial mAb manufacturing, reports the presence of hundreds to thousands of HCP species in the clarified cell culture fluids fed to Protein A adsorbents. In this context, a great deal of attention is focused on a portion of the CHO secretomes formed by a subset of HCPs known in the literature as “problematic” HCPs. These species pose a threat to the patient's health in that they are either responsible for immunogenic responses or for causing degradation of the mAb product. In the context of biomanufacturing, a number of these species co-elute with the mAb product form Protein A adsorbents, thereby charging the subsequent polishing step with the burden of their complete removal. Several of these “problematic” HCPs have been reported to cause delays in clinical trials of mAbs, process approval, and even product withdrawal.

The binding selectivity of peptide ligands for the target IgG is therefore crucial for their effectiveness as Protein A-mimetics. Rapid in silico evaluation of peptide binding to HCP impurities is a powerful potential tool for ligand development prior to laborious experimental evaluation. In this context, we selected a panel of 14 “problematic” HCPs as targets for WQRHGI (SEQ ID NO:1), MWRGWQ (SEQ ID NO:2), RHLGWF (SEQ ID NO:3), and GWLHQR (SEQ ID NO:4) variants for use in a series of docking studies. This panel includes several peroxiredoxins, carboxypeptidases, enolases, glutathione S-transferases, cathepsins, and lipoprotein lipase, as shown in Table 2. Since proteins These available PBD entries from multiple organisms were analyzed in terms of their sequence homology and structural similarity to CHO HCPs. Sequence homology was calculated using the protein sequence alignment tool SIM on ExPASy, whereas structural similarity was calculated using the flexible Java-FATCAT comparison method on the RCSB PDB Protein Comparison Tool. Sequence blasting indicated high homology between proteins of different origin organisms for Peroxiredoxin (sequence identity 68.07%; similarity 83.13%), Glutathione S-transferase (sequence identity 84.7%; similarity 89.5%), Cathepsin B (sequence identity 82.7%; similarity 88.1%), and Cathepsin D (sequence identity 86.8%; similarity 92.4%). Structural similarity between CHO HCP proteins and the selected non-hamster proteins was also very high, as shown by the similarities for Peroxiredoxin (89%), Glutathione S-transferase (100%), Cathepsin B (99%), and Cathepsin D (93.8%).

The crystal structures of these HCPs were analyzed in silico by running a “druggability” assessment using PockDrug to identify putative binding pockets to accommodate linear 9-mer peptides (X₁X₂X₃X₄X₅X₆GSG). This probed the protein surfaces of each HCP to search for peptide binding with appropriate size and shape, exposure to solvent, profiles of hydrophobicity and hydrophilicity, and hydrogen-bonding ability. The number of binding sites on each HCP is described in Table 2. All noted proteins possessed at least 1 and no more than 4 putative binding sites.

In order to dock proteins on putative binding sites, coordinate files of the peptide variants WQRHGI (SEQ ID NO:1), MWRGWQ (SEQ ID NO:2), RHLGWF (SEQ ID NO:3), and GWLHQR (SEQ ID NO:4) were generated via explicit solvent molecular dynamics (MD) simulations in the AMBER 14 simulation suite using the ff14SB force field. Briefly, a 200 ps MD simulation was conducted for every peptide in a simulation box with periodic boundary conditions containing 2,500 water molecules, using the 2 fs time step and applying the LINCS algorithm to constrain all the covalent bonds. The resulting peptide conformations were docked in silico against the putative binding sites on the crystal structures of selected HCPs using the docking software HADDOCK. The resulting poses for every HCP:peptide docking were clustered based on a fraction of common contacts. The peptide-HCP complexes in the clusters containing the highest population of structures were analyzed using scoring function, XScore, to select a final set of binding poses of the peptide variants on each of the 14 HCP targets. These were analyzed using the PRODIGY (PROtein binDIng enerGY prediction) webserver to calculate the corresponding values of binding energy (ΔG_{b(XScore)}). The results were averaged across the different binding sites and the resulting values of the binding energy of peptide binding to HCP (ΔG_{b(XScore)}) are listed in Table 4. To facilitate the comparison between simulated IgG binding and HCP binding by the various peptide variants, the average values of the calculated protein-peptide ΔG_{b(XScore)} and K_{D(XScore)} for both the global HCPs and IgG are reported for all peptides in Table 5.

TABLE 4
Values of average binding energy of the peptide-protein complexes onto a
panel of select HCPs (Lig. shown from left to right: SEQ ID NO: 4; SEQ ID
NO: 2; SEQ ID NO: 3; and SEQ ID NO: 1).

| Lig. | | | | |
|---|---|---|---|---|
| PDB | GWLHQR | MWRGWQ | RHLGWF | WQRHIGI |
| 3HY2 | −4.3 kcal/mol | −4.4 kcal/mol | −4.9 kcal/mol | −3.6 kcal/mol |
| | 7 × 10⁻⁴ M | 6.0 × 10⁻⁴ M | 2.6 × 10⁻⁴ M | 2.3 × 10⁻³ M |
| 3VWU | −3.6 kcal/mol | −3.4 kcal/mol | −6.9 kcal/mol | −3.2 kcal/mol |
| | 2.2 × 10⁻³ M | 3.2 × 10⁻⁴ M | 8.7 × 10⁻⁶ M | 4.5 × 10⁻³ M |
| 2ZS9 | −4.0 kcal/mol | −4.1 kcal/mol | −5.7 kcal/mol | −4.4 kcal/mol |
| | 1.1 × 10⁻³ M | 1.1 × 10⁻² M | 6.6 × 10⁻⁵ M | 5.9 × 10⁻⁴ M |
| 5OM9 | −4.0 kcal/mol | −4.3 kcal/mol | −6.8 kcal/mol | −4,2 kcal/mol |
| | 1.1 × 10⁻³ M | 7.0 × 10⁻⁴ M | 1,0 × 10⁻⁵ M | 8.3 × 10⁻⁴ M |
| 2PSN | −3.1 kcal/mol | −3.3 kcal/mol | −5.0 kcal/mol | −3.7 kcal/mol |
| | 6.3 × 10⁻³ M | 3.8 × 10⁻³ M | 2.2 × 10⁻⁴ M | 1.9 × 10⁻³ M |
| 5J41 | −4.5 kcal/mol | −4.9 kcal/mol | −6.1 kcal/mol | −3.6 kcal/mol |
| | 5.0 × 10⁻⁴ M | 2.6 × 10⁻⁴ M | 3.4 × 10⁻⁶ M | 2.3 × 10⁻³ M |
| 3O76 | −4.9 kcal/mol | −5.7 kcal/mol | −6.9 kcal/mol | −4.2 kcal/mol |
| | 2.6 × 10⁻⁴ M | 6.6 × 10⁻⁵ M | 8.7 × 10⁻⁶ M | 8.3 × 10⁻⁴ M |
| 5MBL | −3.7 kcal/mol | −4.5 kcal/mol | −4.2 kcal/mol | −3.6 kcal/mol |
| | 1.9 × 10⁻³ M | 5.0 × 10⁻⁴ M | 8.3 × 10⁻⁴ M | 2.3 × 10⁻³ M |
| 1THE | −4.3 kcal/mol | −4.2 kcal/mol | −6.0 kcal/mol | −4.2 kcal/mol |
| | 7.0 × 10⁻⁴ M | 8.3 × 10⁻⁴ M | 4.0 × 10⁻⁵ M | 8.3 × 10⁻⁴ M |
| 4OD9 | −3.9 kcal/mol | −4.6 kcal/mol | −6.3 kcal/mol | −3.1 kcal/mol |
| | 1.5 × 10⁻⁴ M | 4.2 × 10⁻⁴ M | 1.3 × 10⁻⁴ M | 5.3 × 10⁻³ M |
| 5UX4 | −4.9 kcal/mol | −3.3 kcal/mol | −6.5 kcal/mol | −3.4 kcal/mol |
| | 2.6 × 10⁻⁴ M | 3.8 × 10⁻³ M | 1.7 × 10⁻⁵ M | 3.2 × 10⁻³ M |
| 5MAE | −4.8 kcal/mol | −4.7 kcal/mol | −6.3 kcal/mol | −4.2 kcal/mol |
| | 5.6 × 10⁻⁵ M | 3.6 × 10⁻⁴ M | 2.4 × 10⁻⁵ M | 8.3 × 10⁻⁴ M |
| 3MN8 | −4.0 kcal/mol | −4.1 kcal/mol | −4.4 kcal/mol | −3.7 kcal/mol |
| | 1.2 × 10⁻⁴ M | 9.9 × 10⁻⁴ M | 6.0 × 10⁻⁴ M | 1.9 × 10⁻³ M |
| 6E7K | −4.3 kcal/mol | −3.9 kcal/mol | 6.1 kcal/mol | −3.9 kcal/mol |
| | 7.0 × 10⁻⁴ M | 1,4 × 10⁻³ M | 3.3 × 10⁻⁵ M | 1.4 × 10⁻² M |

TABLE 5
Values of average binding energy of the peptide binding (Lig.
shown from top to bottom: SEQ ID NO: 1; SEQ ID NO: 2; SEQ ID
NO: 3; and SEQ ID NO: 4).

| | HCP | | IgG | |
|---|---|---|---|---|
| PDB | ΔG_{b(χ Score)} | K_{D(χ Score)} | ΔG_{b(χ Score)} | K_{D(χ Score)} |
| Lig. | (kcal/mol) | (M) | (kcal/mol) | (M) |
| WQRHGI | −4.15 | 9.0 × 10⁻⁴ | −6.6 | 7.8 × 1⁰⁻⁶ |
| MWRGWQ | −4.24 | 7.8 × 10⁻⁴ | −6.8 | 1.0 × 10⁻⁸ |
| RHLGWF | −5.79 | 5.7 × 10⁻⁵ | −7.6 | 2.7 × 10⁻⁶ |
| GWLHQR | −3.78 | 1.6 × 10⁻³ | −6.3 | 2.4 × 10⁻⁴ |

The predicted K_{D(XScore)} of peptides interacting with HCPs were at least one order of magnitude higher than that for IgGs. Explicit atomistic simulations were also performed to predict binding of peptide to HCPs using the AMBER15 package, but after multiple simulations found that none of the purported binding sites would accommodate the 4 peptides. These atomistic studies confirm the docking energy predictions that the peptides will likely not bind HCPs in an appreciable amount.

Variants WQRHGI (SEQ ID NO:1) and MWRGWQ (SEQ ID NO:2) provide the appropriate balance between binding strength for IgG and selectivity (ΔG_{bXScore;IgG}/ΔG_{bXScore;HCP}) and were therefore selected for further experimental characterization. In the docking study using HCPs, WQRHGI (SEQ ID NO:1), MWRGWQ (SEQ ID NO:2), and GWLHQR (SEQ ID NO:4) showed low binding affinity towards all selected HCPs. As well, GWLHQR (SEQ ID NO:4) was predicted to have the lowest affinity for IgG. Based on the K_{b(XScore)} for the binding of variant RHLGWF (SEQ ID NO:3) to HCPs from initial docking studies, RHLGWF (SEQ ID NO:3) was expected to have a comparatively poor selectivity despite its high binding strength for IgG. Additional considerations that led to variant WQRHGI's (SEQ ID NO:1) selection for experimental characterization included in silico predictions of low binding energy and specific affinity for IgG. MWRGWQ (SEQ ID NO:2) was chosen for its resemblance to the reference sequence HWRGWV (SEQ ID NO:18).

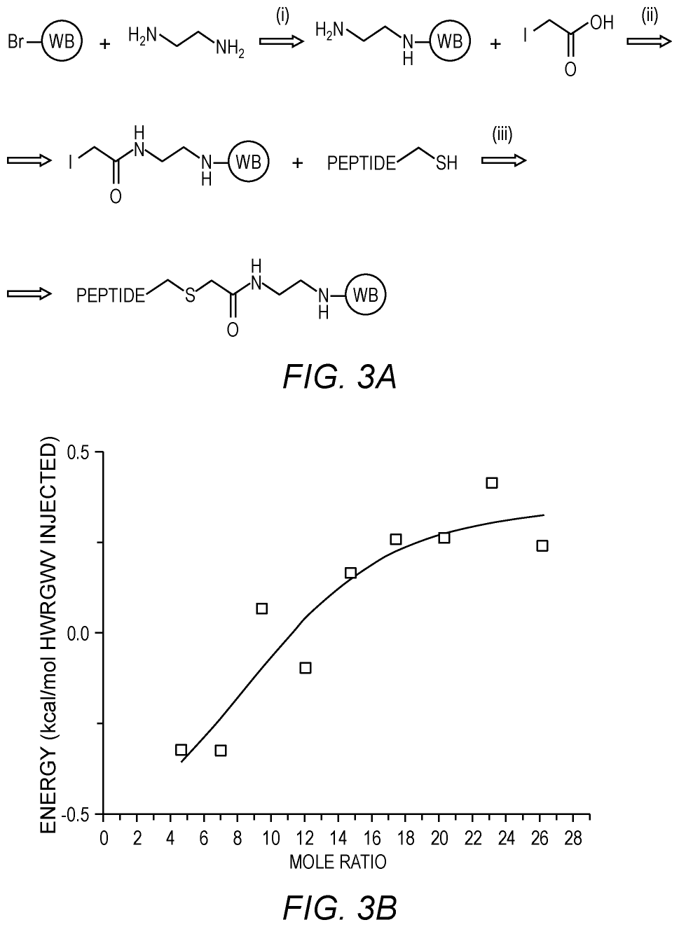

Characterization of binding affinity for IgG-binding peptide variants WQRHGI (SEQ ID NO:1) and MWRGWQ (SEQ ID NO:2) in non-competitive conditions: Candidate peptide ligands WQRHGI (SEQ ID NO:1) and MWRGWQ (SEQ ID NO:2) were selected for experimental evaluation of IgG binding in non-competitive conditions (pure IgG in solution). The cysteine-derivatized sequences WQRGHIC (SEQ ID NO:32) and MWRGWQC (SEQ ID NO:31) were synthesized, purified, and conjugated to iodacetyl-activated TREN WorkBeads (WB) resins (FIG. 3A). Iothermal titration calorimetry (ITC) tests conducted by titrating WQRHGI (SEQ ID NO:1) in solution against human polyclonal IgG using a Nano ITC Low Volume calorimeter confirmed that the binding energy of the peptide to target protein IgG was low enough for specific binding (K_{D(ITC)} of 5.88×10⁻⁵ M, which indicates a moderate affinity). Briefly, ten 5 μL injections of a 2 mg/mL solution of each peptide in PBS were performed in 300 mL of 5 mg/mL solution of polyclonal IgG in PBS, while maintaining the temperature constant at 25° C. The titration data were analyzed using NanoAnalyze (TA Instruments) and plotted using an “independent fitting.” This fit the resultant Wiseman plot with parameters corresponding to a non-competitive single-site binding phenomenon to calculate the binding affinity and the stoichiometry, which is defined as the number of interacting peptides per IgG (N) of the interaction (FIG. 3B). A constant blank was also utilized in the fitting to account for the heat of dilution of the IgG substrate. The integration of the energy peaks returned a K_{D(ITC)} of 5.88×10⁻⁵ M and a stoichiometry of 10 for WQRHGI (SEQ ID NO:1).

The difference between the values of K_{D(Solid)} predicted on solid phase (3.2×10⁻⁶ M) and value of K_{D(ITC)} obtained via ITC (5.88×10⁻⁵ M) can be explained by accounting for the formation of peptide aggregates, namely physical dimers and trimers, that were likely formed as the peptide concentration in solution increases with the number of injections. Evidence for this is the appearance of the endothermic peaks at the end of the titration (FIG. 3C). Peptide aggregation as an endothermic phenomenon has been reported numerous times in the literature. These self-assembled peptide dimers and trimers are likely to have a lower affinity for IgG compared to the peptide monomers. This could explain their effectively higher K_{D} (lower affinity) compared to the in silico studies, which assume the peptide ligand to always be in a monomeric state. It also accounts for the high molarity of binding.

MWRGWQ's (SEQ ID NO:2) binding affinity could not be examined using ITC. When in solution, peptide MWRGWQ (SEQ ID NO:2) exhibited strong self-associative properties and tended to gel at neutral pH, but could be dissolved at a lower pH. However, when the peptide was dissolved in a lower pH solution, the heat of mixing between the different pH solutions was extremely high, and peptide-peptide or peptide-IgG binding energies upon titration became difficult to differentiation from the heat of mixing in ITC experiments.

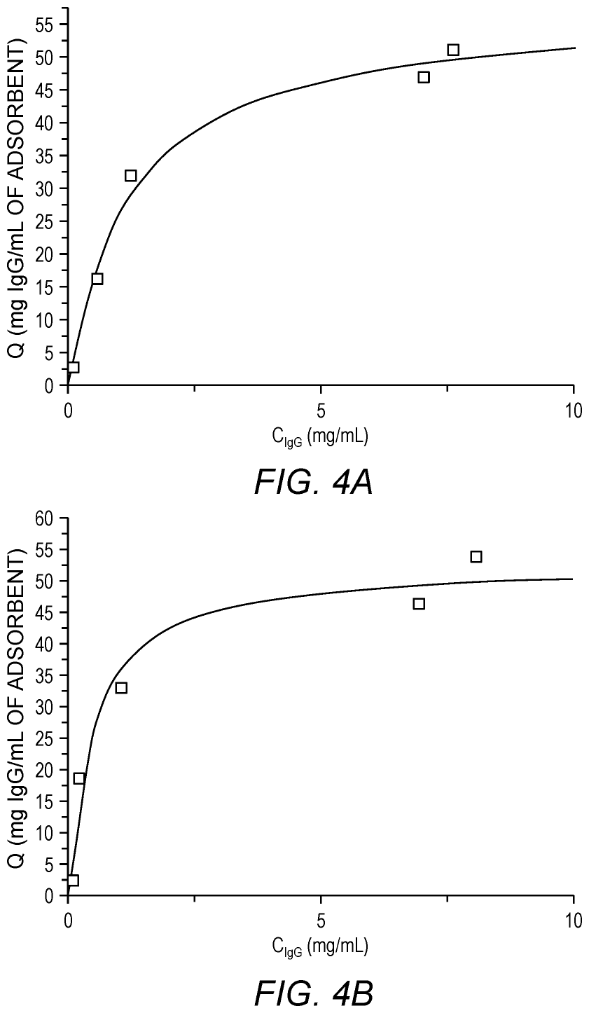

Isothermal adsorption studies determined a K_{D(Solid)} of 3.2×10⁻⁶ and Qₘₐₓ of 52.6 mg IgG/mL resin for WQRGHIC (SEQ ID NO:32)—WorkBeads and a K_{D(Solid)} of 8.1 10 ⁻⁶ and Qₘₐₓ of 57.5 mg IgG/mL resin for MWRGWQC (SEQ ID NO:31)—WorkBeads. These results indicate that the sequences found through an in silico screen are, in fact, good binders of IgG. Each 30 μL aliquot of adsorbent was equilibrated in binding buffer (PBS, pH 7.4), and incubated with 200 μL of IgG solution at increasing concentrations over a range of 0-10 mg/mL, at room temperature for 2.5 hours. The amount of unbound IgG was determined by analyzing the supernatants via Micro BCA Protein Assay Kit. The amount of bound IgG per volume of resin (Q) was determined by mass balance and plotted against the corresponding equilibrium concentration of unbound IgG in solution (C_{I}gG). The data were fit to a Langmuir isotherm model, thus providing a value of maximum binding capacity (Qₘₐₓ) and dissociation constant (K_{D}). The adsorption isotherms of IgG on WQRGHIC (SEQ ID NO:32)—WorkBeads and MWRGWQC (SEQ ID NO:31)—WorkBeads are reported in FIG. 4A and 4B, respectively.

The values of K_{D(Solid)} obtained by Langmuir fitting (Table 6) were lower than the value calculated using ITC (FIG. 3B) for WQRHGI (SEQ ID NO:1), indicating a stronger effective affinity on solid phase. This can be explained by considering that multiple ligands displayed on the chromatographic resin can bind a single IgG target. As a symmetrical dimer, in fact, the Fc region of IgG contains at least two binding sites for each ligand. The cooperative binding by multiple ligands results in a higher binding strength—a phenomenon known as “avidity”—during protein adsorption. It is worth noting that, despite the more moderate affinity of the peptide ligands in comparison to Protein A, the values of Qₘₐₓ also compare well with those obtained in prior work with HWRGWV (SEQ ID NO:18) (Naik et al. 2011 J. of Chromatography A 1218(13) : 1691-1700; Kish et al. 2013 Industrial and Engineering Chemistry Research 52(26):8800-8811) and are reasonable when compared with Protein A adsorbents (Hahn et al. 2003 Adsorption J. of the Int. Adsorption Society 790:35-51). This high capacity was attributed to the high density of the peptide ligands, which at 100 milliequivalents/mL was likely high enough to allow multiple ligand interactions per adsorbed IgG molecule.

TABLE 6
Values of dissociation constant and static
binding capacity of MWRGWQ (SEQ ID NO: 2)-
Workbeads and WQRHGI (SEQ ID NO: 1)-Workbeads
adsorbents obtained by fitting IgG adsorption
data to a Langmuir model.

| Ligand | Qₘₐₓ (mg IgG/mL resin) | K_{D(Solid)} (M) |
|---|---|---|
| MWRGWQ | 57.5 | 8.1 × 10⁻⁶ |
| WQRHGI | 52.6 | 3.2 × 10⁻⁶ |

A limited library of residue-by-residue changes confirmed the importance of each residue in peptides WQRHGI (SEQ ID NO:1) and MWRGWQ (SEQ ID NO:2) in reducing the binding energy between the peptide and the IgG target. Further, these results supported in silico predictions of the relative importance of each residue as seen in FIG. 2. This was accomplished by designing and constructing an ensemble of 20 variants of peptides WQRHGI (SEQ ID NO:1) and MWRGWQ (SEQ ID NO:2). Selected residues in positions 1-6 were mutated. The peptide variants were synthesized directly on Toyopearl AF-amino-650M resin via Fmoc/tBu chemistry. The resulting adsorbents were incubated with a solution of human IgG at 2 mg/mL at a ratio of 1 mL of resin per 3.5 mL of solution for 30 min at room temperature. The residual concentration of IgG in solution was determined by Bradford concentration assay of the supernatants and utilized to calculate the amounts of bound IgG per volume of resin; Table 7 reports the % binding, defined as mg IgG bound by variant/mg IgG bound by original sequence (either WQRHGI (SEQ ID NO:1) or MWRGWQ (SEQ ID NO:2))×100%, of each sequence variant. This shows the importance of each residue in maintain binding strength and, thus, reducing binding energy.

TABLE 7
Values of IgG binding for variants of peptides WORHGI (SEQ ID NO: 1) and
MWRGWQ (SEQ ID NO: 2). Sequences as shown from top to bottom: SEQ ID NO: 2,
SEQ ID NO: 23, SEQ ID NO: 8, SEQ ID NO: 21, SEQ ID NO: 24, SEQ ID NO: 25,
SEQ ID NO: 5, SEQ ID NO: 6, SEQ ID NO: 7, SEQ ID NO: 26, SEQ ID NO: 22, SEQ
ID NO: 1, SEQ ID NO: 29, SEQ ID NO: 12, SEQ ID NO: 27, SEQ ID NO: 28, SEQ
ID NO: 11, SEQ ID NO: 33, SEQ ID NO: 10, and SEQ ID NO: 9.

| Sequence | 1 | 2 | 3 | 4 | 5 | 6 | % Binding |
|---|---|---|---|---|---|---|---|
| MWRGWQ | M | W | R | G | W | Q | 100.00% |
| ΛWRGWQ | Λ | W | R | G | W | Q | 74.66% |
| GWRGWQ | G | W | R | G | W | Q | 78.37% |
| MFRGWQ | M | F | R | G | W | Q | 56.75% |
| MGRGWQ | M | G | R | G | W | Q | Undetected |
| MWχGWQ | M | W | χ | G | W | Q | 0.48% |
| MWRAWQ | M | W | R | A | W | Q | 96.96% |
| MWRWQ | M | W | R | | W | Q | 91.55% |
| MWRGEQ | M | W | R | G | F | Q | 89.19% |
| MWRGGQ | M | W | R | G | G | Q | 26.00% |
| MWRGWN | M | W | R | G | W | N | 18.90% |
| WQRHGI | W | Q | R | H | G | I | 100.00% |
| FQRHGI | F | Q | R | H | G | I | 37.18% |
| WNRHGI | W | N | R | H | G | I | 77.43% |
| WQχHGI | W | Q | χ | H | G | I | 0.95% |
| WQRAGI | W | Q | R | A | G | I | 62.80% |
| WQRHAI | W | Q | R | H | A | I | 95.43% |
| WQRHI | W | Q | R | H | | I | 86.89% |
| WQRHGV | W | Q | R | H | G | V | 96.04% |
| WQRHGL | W | Q | R | H | G | L | 99.09% |

*Λ represents Nor-Leucine; χ represents Citrulline

The variants produced by replacing residues that were predicted to impact binding strength unfavorably (M in MWRGWQ (SEQ ID NO:2)) or negligibly (G in MWRGWQ (SEQ ID NO:2); Q and G in WQRHGI (SEQ ID NO:1)) showed minimal loss of IgG binding. Worthy of notice was the deletion of G which, consistently with its calculated contribution, resulted in a negligible decrease in IgG binding. On the other hand, the replacement of residues predicted to be critical for IgG binding, such as W in WQRHGI (SEQ ID NO:1), Wi in MWRGWQ (SEQ ID NO:2), R in both peptides, and H in WQRHGI (SEQ ID NO:1), resulted in major loss of IgG yield, as expected. In particular, the positive charge displayed by R was found to be critical towards binding, since its replacement with Citrulline (Cit) completely obliterated peptide binding. This is understandable since the side chain functional groups on Cit and R feature highly similar molecular structure and hydrogen-bonding ability but differ in charge, the ureyl-group on Cit being neutral and the guanidyl group on R being positively charged at neutral pH. Finally, residue 6 did not follow predicted trends regarding its importance for binding with either peptide. The replacement of Q in MWRGWQ (SEQ ID NO:2), which was expected to minimally alter binding affinity, caused a major loss in IgG yield, whereas the replacement of Ile in WQRHGI (SEQ ID NO:1), which was expected to result in a major loss in IgG binding, resulted in inconsequential losses.

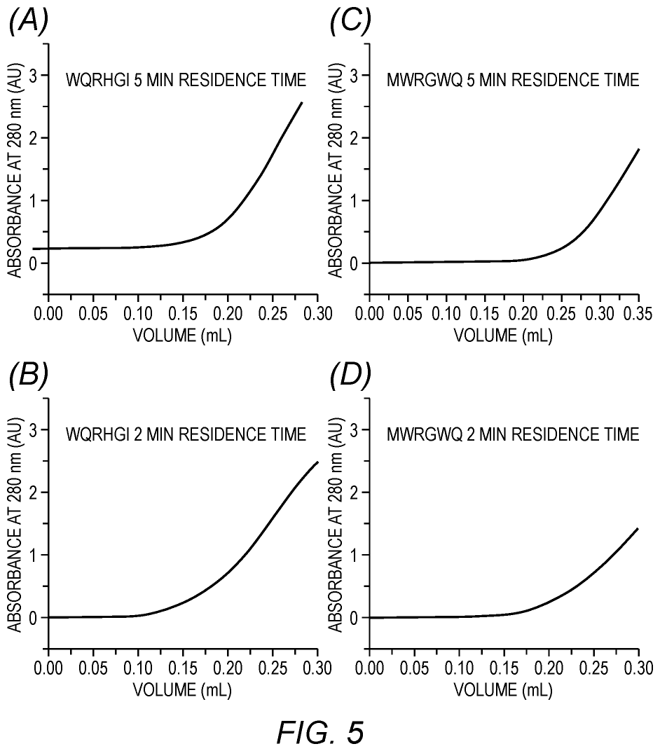

The values of the dynamic binding capacity (DBC) of IgG were measured for MWRGWQC (SEQ ID NO:31)—WorkBeads and WQRGHIC (SEQ ID NO:32)—WorkBeads by breakthrough assays and found to be comparable to the DBC of other peptide ligands for IgG. Breakthrough curves (FIG. 5 panels A-D) were obtained by flowing a 20 mg/mL solution of IgG in PBS through the WQRGHIC (SEQ ID NO:32)-WB and MWRGWQC (SEQ ID NO:31)-WB adsorbents at two different flow rates (0.05 and 0.02 mL/min) corresponding to two different residence times (2 and 5 minutes). Similar to what was observed in static experiments, MWRGWQC (SEQ ID NO:31)—WorkBeads showed a slightly higher binding capacity than WQRGHIC (SEQ ID NO:32)-WB, but both were similar to HWRGWVC (SEQ ID NO:34)—WorkBeads (Table 8). In terms of binding capacity, both WQRHGI (SEQ ID NO:1) and MWRGWQ (SEQ ID NO:2) proved to be credible alternatives to Protein A and other IgG binding ligands.

```
TABLE 8
Values of dynamic binding capacity at 10%
breakthrough obtained from breakthrough curves
  in FIGS. 4A-4B (Resin sequences shown from top
to bottom: SEQ ID NO: 1, SEQ ID NO: 2, and
  SEQ ID NO: 34).
```

| | Residence | DBC |
|---|---|---|
| Resin | Time(min.) | (mg IgG/mL resin) |
| WORHGI | 5 | 43.8 |
| | 2 | 33.6 |
| MWRGWQ | 5 | 55.3 |
| | 2 | 44 |
| HWRGWV[77] | 5 | 36 |
| | 2 | 25 |

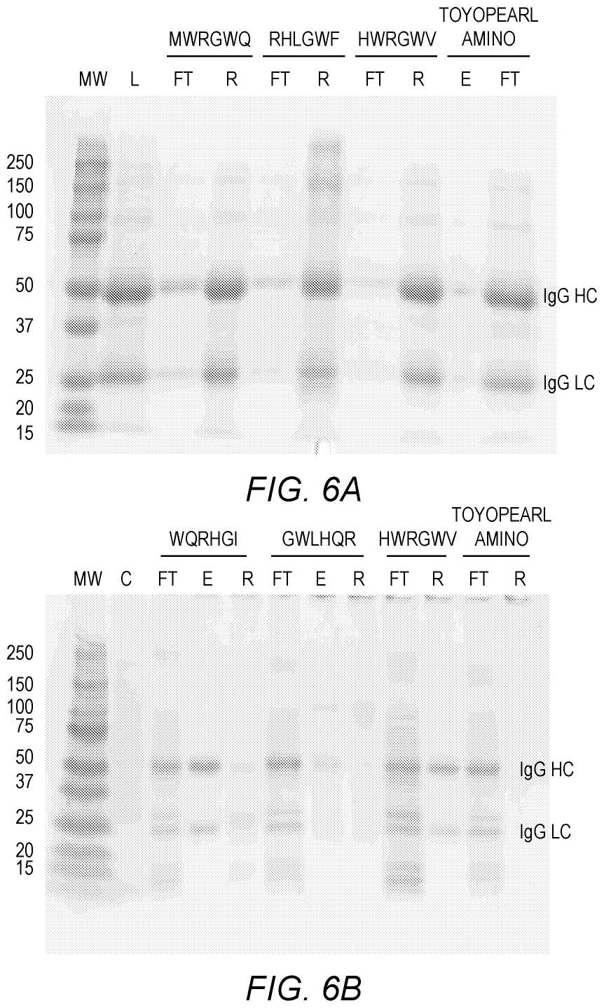

Characterization of IgG-binding peptide variants WQRHGI (SEQ ID NO:1), MWRGWQ (SEQ ID NO:2), RHLGWF (SEQ ID NO:3), and GWLHQR (SEQ ID NO:4) in competitive conditions: The four selected sequence variants were tested for their ability to purify human IgG from a CHO cell culture supernatant and found largely to mirror their in silico predictions. Even though they seemed to underperform in silico, RHLGWF (SEQ ID NO:3) and GWLHQR (SEQ ID NO:4) were tested alongside WQRHGI (SEQ ID NO:1) and MWRGWQ (SEQ ID NO:2) in these conditions in order to confirm their ability to bind IgG and examine their selectivity as predicted in silico. The feedstock was prepared by spiking human polyclonal IgG into a clarified null CHO-S cell culture fluid to obtain an IgG concentration of 1 mg/mL and a CHO HCP concentration of 0.205 mg/mL. An aliquot of 500 μL was loaded onto each peptide adsorbent in static conditions for 30 min. Following a washing step with PBS to remove loosely bound proteins, a first elution step was conducted using 0.1 M glycine buffer pH 2.5 to remove all bound proteins. Flow through fractions and pH 2.5 elution fractions were loaded neat and analyzed by SDS PAGE (FIGS. 6A-6B). The values of IgG purity in the eluted fractions were determined by densitometric analysis of the corresponding lanes on the gels, and are reported in Table 9. The values were calculated by densitometric analysis of the SDS-PAGEs reported in FIGS. 6A-6B.

TABLE 9
Values of IgG purity in the elution fractions
(E, pH 4) and regeneration fractions (R, pH 2.5)
expressed as % value of eluted IgG over total
eluted proteins. Resin sequences as shown
from top to bottom: (Gel A) SEQ ID NO 2, SEQ ID
NO: 3, SEQ ID NO: 18; (Gel B) SEQ ID NO: 1,
SEQ IS NO: 4, SEQ ID NO: 18.

| Gel | Resin | Lane | % Purity |
|---|---|---|---|
| A | — | F | 95.10% |
| | MWRGWQ | FT | 98.42% |
| | | R | 97.82% |
| | RHLGWF | FT | 100.00% |
| | | R | 52.28% |
| | HWRGWV | FT | 100.00% |
| | | R | 97.81% |
| | Toyopearl Amino | R | 92.08% |
| | | FT | 96.87% |
| B | — | C | 0.00% |
| | WQRHGI | FT | 52.71% |
| | | E | 100.00% |
| | | R | 78.79% |
| | GWLHQR | FT | 57.02% |
| | | E | 100.00% |
| | | R | 0.00% |
| | HWRGWV | FT | 45.40% |
| | | R | 93.79% |
| | Toyopearl Amino | FT | 62.15% |
| | | R | 0.00% |

As predicted by computational studies, peptides GWLHQR (SEQ ID NO:4) and WQRHGI (SEQ ID NO:1) returned the highest values of IgG purity in the eluted fractions, both an apparent 100% even in the face of highly sensitive silver staining techniques. These results corroborate the low-to-no binding of GWLHQR (SEQ ID NO:4) and WQRHGI (SEQ ID NO:1) for CHO HCPs indicated by the in silico binding studies. The GWLHQR(SEQ ID NO:4)-based adsorbent, however, afforded a lower IgG yield, indicating low binding capacity. Experimental work, in this instance, did not validate GWLHQR (SEQ ID NO:4) as a potential binder of IgG. This can be expected, since the computational search algorithm was used to limit the number of potential peptide variants to bind IgG. Since atomistic simulation tends to result in relative binding energies, this is not an entirely unexpected result. As a result of poor in vitro binding strength, GWLHQR (SEQ ID NO:4) was not further pursued.

Variant RHLGWF (SEQ ID NO:3) afforded high IgG yield but very low IgG purity (52.28%), and was thus not pursued in further studies. This was consistent with the in silico results, which showed substantial binding of this peptide to the majority of the HCPs in the selected panel. This result was attributed to the higher hydrophobicity of RHLGWF (SEQ ID NO:3) compared to GWLHQR (SEQ ID NO:4) and WQRHGI (SEQ ID NO:1), which promotes non-specific protein binding. To quantitatively compare the hydrophobicity of these peptides, their Grand Average of Hydropathy (GRAVY) index was calculated utilizing the algorithm developed by Kyte and Doolittle (1982 J. of Molecular Biology 157(1):105-132) wherein a higher (or less negative) score indicates higher hydrophobicity. The GRAVY index of RHLGWF (SEQ ID NO:3) was 0.4, that of GWLHQR (SEQ ID NO:4) was −1.45, and that of WQRHGI (SEQ ID NO:1) was −0.82. In general, higher GRAVY indexes indicate higher hydrophobicity, which can lead to nonspecific binding.

Issues with resin reusability due to oxidation of the methionine in peptide variant MWRGWQ (SEQ ID NO:2) led us to eliminate the sequence from further studies. This was disappointing since MWRGWQ (SEQ ID NO:2) demonstrated high binding selectivity for IgG—in line with the in silico predictions—affording a value of IgG purity of 97.82%. It was also noted that, with a GRAVY index of −1.38, MWRGWQ (SEQ ID NO:2) supports the correlation tying low HCP binding to lower GRAVY scores. Methionine, however, is prone to oxidation to methionine sulfoxide (MetO) in the presence of mild oxidants; these include the acid environments (pH 4 and pH 2.5) utilized for protein elution and regeneration of the adsorbents. Thus, methionine containing peptide ligands are likely to undergo slow oxidation upon extensive reuse, resulting in loss of IgG binding affinity. This explains why the MWRGWQ (SEQ ID NO:2) resin was not reliably reusable over several chromatographic purification runs, which severely limits its usefulness in industrial processes.

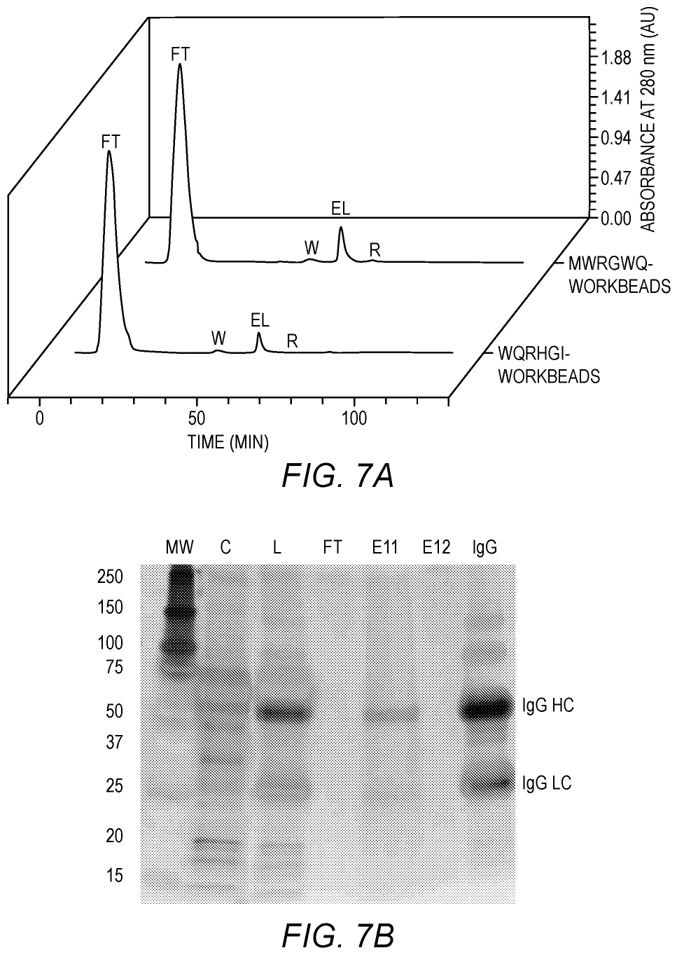

The high purity of the recovered IgG using WQRHGI (SEQ ID NO:1), as calculated by densitometric analysis (100%) was confirmed by the HCP LRV value of 2.7, thus indicating WQRHGI (SEQ ID NO:1) has purification abilities similar to Protein A. This is a remarkable result. To the best of our knowledge, WQRHGI (SEQ ID NO:1) exhibits the highest HCP LRV ever reported for small synthetic peptide ligands, including that of the reference sequence, HWRGWV (SEQ ID NO:18), which provided an optimized LRV of 1.6. The high product purity is a consequence of the high binding specificity of the peptide ligand as well as the additional washing step. In a competitive, mobile phase experiment, a volume of 0.5 mL of feedstock solution of IgG in CHO cell culture fluid was injected in a 0.1 mL column packed with WQRHGI (SEQ ID NO:1)-WB resin at a 5 min residence time. Elution buffers remained as 0.2 M acetate buffer at pH 4 and 0.1 M glycine buffer at pH 2.5. The washing step (0.1 M additional NaCl in PBS, pH 7.4) removes a small amount of HCP impurities, which shows the importance of a high-salt wash to reduce non-specifically bound impurities (FIG. 7A). The collected chromatographic fractions were analyzed by SDS-PAGE (FIG. 7B, silver stained to highlight diluted CHO HCPs). The % values of IgG in the fractions (expressed as a ratio of IgG concentration over total protein (e.g., IgG+CHO HCPs)) were calculated by densitometric analysis of the lanes in the SDS gel and were as follows: Control (C), 0.00%; Load (L) 59.77%; Flowthrough (FT), 0.00%; Elution 1 (El1), 100.00%; Elution 2 (El2), 0.00%; IgG 93.30%.

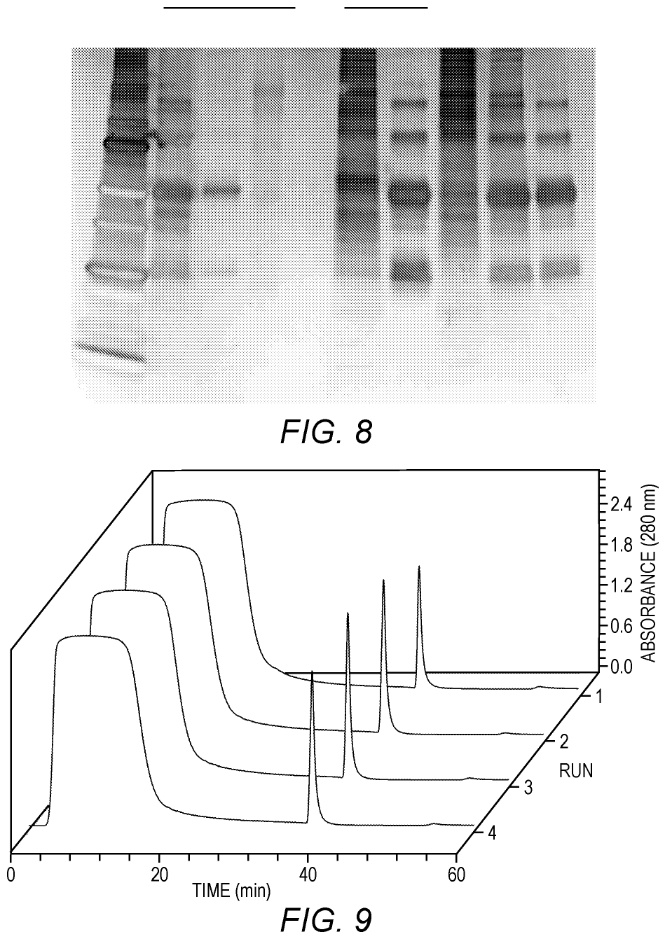

Using a ligand density lower than reported in the previous section, WQRHGI (SEQ ID NO:1)—WorkBeads afforded 99.7% of the HCP clearance obtained with Hi-Trap Protein A resin, further indicating that our peptide resin is comparable in selectivity to Protein A. Since higher ligand density can often lead to increased non-specific interactions, an adsorbent with reduced ligand density was produced by lowering the ligand density from 100 milliequivalents/mL of WB resin to 35.2 milliequivalents/mL. The resulting adsorbent was challenged against the same CHO feedstock as before (1 mg/mL IgG combined with 0.205 mg/mL CHO HCPs). Following adsorption in PBS, the resin was washed with PBS, after which the bound proteins were eluted with 0.2 M acetate buffer pH 4. The flow-through, elution, and regeneration fractions were collected and analyzed by SDS-PAGE (FIG. 8) and by CHO HCP-specific ELISA to determine the ratio between the HCP LRV provided by the WQRHGI (SEQ ID NO:1)—WorkBeads and that provided by Protein A resin. The purity of eluted IgG obtained by electrophoretic analysis using sensitive silver staining was measured at 100%. Silver staining was adopted to magnify the presence of protein impurities coeluted with IgG. Densitometric analysis of the gel could not in fact detect any protein species other than the heavy and light chains of human IgG. Table 10 shows % values of IgG in the chromatographic fractions expressed as ratio of IgG over total protein (IgG+CHO HCPs). The values were calculated by densitometric analysis of the SDS-PAGEs reported in FIG. 8.

TABLE 10
% values of IgG from FIG. 8, including WQRHGI
(SEQ ID NO: 1)-WorkBeads.

| Resin | Lane | % Purity |
|---|---|---|
| WQRHGI-WorkBeads | FT | 55.19% |
| | EI | 100.00% |
| Protein A | FT | 55.74% |
| | E | 100.00% |
| CHO | CHO | 0.00% |
| Load | Ld | 67.98% |
| IgG | IgG | 100.00% |

Adsorbent WQRHGI (SEQ ID NO:1)—WorkBeads was also shown to be reusable. The WQRHGI (SEQ ID NO:1)—WorkBeads adsorbent was challenged with repeated cycles of IgG purification from the CHO cell culture supernatant. Specifically, 4 cycles were repeated wherein WQRHGI (SEQ ID NO:1)-WB was contacted with the CHO fluid containing human IgG at 1 mg/mL at a residence time of 5 minutes, washed with PBS, owed with 0.2 M acetate buffer pH 4 to elute the bound IgG, regenerated with 0.1 M glycine buffer pH 2.8, and finally washed with 1% acetic acid. As seen in FIG. 9, the resin did not show any decrease in binding performance over the 4 cycles.

Multiple Protein A alternatives are available, but none boast clearances high enough to be called true mimetics. As a class of molecules, peptides can be synthesized synthetically, which reduces the chance of contamination by disease-causing particles and reduces batch-to-batch variation. With a wide range of available sequence space, peptides exhibit an enormous variety of conformations and functions that can be taken advantage of. Several peptide ligands have been invented with similar clearances, binding capacities, and purification qualities (Kan et al. 2016 J. of Chromatrography A 1466:105-112; Yang et al. 2009 J. of Chromatography A 1216(6):910-918; Lund et al. 2012 J. of Chromatography A 1225:158-167; Zhao et al. 2014 J. of Chromatography A 1355:107-114; Xue et al. 2016 Biochemical

Engineering Journal 2017:18-25), but the elusive goal of offering a process sufficient to compete with Protein A remains elusive. Non-peptide ligands exist, such as triazine based MAbSorbent A1P and A2P from Prometic Biosciences (Newcombe et al. 2005 J. of Chromatography B 755:37-46; Guerrier et al. 2001 J. of Chromatography B 755:37-46) or GE Healthcare's MEP (Ngo and Khatter, 1990 J. Chromatography 510:2841-291), but none have quite reached the apex of Protein A's HCP clearance.

Herein, computational programs previously shown to improve strength of peptide binding were used to mutate the sequence of peptide HWRGWV (SEQ ID NO:18). Peptide HWRGWV (SEQ ID NO:18) has been extensively shown to bind tightly and specifically to the Fc portion of IgG. The computational program was able to identify several sequences with high in silico predicted affinity to IgG. Using a Monte-Carlo based computational mutation method, a broad range of computational sequence space was investigated. Atomistic MD studies were conducted to show binding of 4 peptides to human IgG, and these same peptides were tested in a novel negative screen against an array of “problematic” HCPs. These combined results indicated 3 of these 4 peptides would bind IgG specifically. In in vitro studies informed by in silico results, three of the four selected sequences exhibited similar but slightly reduced affinity to CHO HCP impurities when compared with the original ligand, HWRGWV (SEQ ID NO:18). However, as predicted by the negative in silico screen, three of the four selected sequences also exhibited lower average affinity for select “problematic” HCPs in initial docking studies and did not bind during MD simulations. These results indicated that these select sequences could effectively separate IgG from cell culture solution.

Studies conducted with IgG and conjugated WQRHGI (SEQ ID NO:1)—WorkBeads and MWRGWQ (SEQ ID NO:2)—WorkBeads showed that these two ligands exhibit similar binding affinity as HWRGWV (SEQ ID NO:18). Each had K_{D(Solid)} values in the micromolar range. Resins WQRHGI (SEQ ID NO:1)—WorkBeads and MWRGWQ (SEQ ID NO:2)—WorkBeads also showed binding capacities similar to that of earlier HWRGWV (SEQ ID NO:18)-based resins and in a range similar to that of several Protein A resins. WQRHGI (SEQ ID NO:1)—WorkBeads is, to date, the best peptide-based ligand alternative to Protein A resins in terms of HCP clearance. Experiments in the presence of CHO proteins validate the MD simulations and docking studies conducted here to predict the reduction of cell culture impurities. As predicted by in silico studies, competitive binding studies showed sequence RHLGWF (SEQ ID NO:3) bound several impurities. While GWLHQR (SEQ ID NO:4) bound few impurities, it also failed to bind the IgG target protein at a high enough yield. MWRGWQ (SEQ ID NO:2) and WQRHGI (SEQ ID NO:1), however, were both capable of binding IgG while simultaneously allowing HCP proteins to pass, as predicted in silico. Using a WQRHGI (SEQ ID NO:1) resin with similar binding capacities to that of previously investigated HWRGWV (SEQ ID NO:18) adsorbents, this study was able to afford HCP clearance greater than 99%; this is unprecedented among synthetic ligands and only attainable with Protein A based resins. This study further showed that WQRHGI (SEQ ID NO:1) resin was reusable with little degradation of performance. The use of a peptide design algorithm to determine target-binding proteins along with MD simulations and docking studies against problematic host-cell proteins could be beneficial when looking for peptide ligands that could specifically bind other targets. Unless a peptide exhibits high levels of hydrophobicity or charge, it is difficult to determine a priori whether a certain peptide sequence will exhibit specificity. The computational methods described here have been shown to correlate well with experimental results in this example with IgG as a binding target. This method discovered two high performing resins, one of which was competitive with industrial standard Protein A by providing 99.7% of the HCP removal provided by a Protein A HiTrap column. This procedure shows great promise for identifying other highly specific ligands, based on both known peptide ligands and for proteins with not-yet-discovered binders.

The foregoing is illustrative of the present invention, and is not to be construed as limiting thereof. The invention is defined by the following claims, with equivalents of the claims to be included therein.

That which is claimed is:

1. A synthetic peptide comprising an amino acid sequence of any one of SEQ ID NOs:1-17 or an amino acid sequence having at least 80%, 85%, 90%, or 95% sequence identity to the amino acid sequence of any one of SEQ ID NOs:1-17.

2. The peptide of claim 1, wherein the peptide has or is configured to provide a host cell protein (HCP) logarithmic removal value (LRV) of at least 2.0, 2.1, 2.2., 2.3, 2.4, 2.5, 2.6, 2.7, 2.8, 2.9, 3.0, 3.1, 3.2, 3.3, 3.4, 3.5, or more as measured by a HCP-specific quantification assay, optionally wherein the peptide has or is configured to provide a HCP LRV of at least 2.5.

3. The peptide of claim 1 or 2, wherein the peptide comprises the amino acid sequence of SEQ ID NO:1, optionally wherein the peptide further comprises a linking amino acid residue (e.g., a cysteine residue or lysine residue) as the C-terminal amino acid residue.

4. The peptide of claim 1 or 2, wherein the peptide comprises the amino acid sequence of SEQ ID NO:2, optionally wherein the peptide further comprises a linking amino acid residue (e.g., a cysteine residue or lysine residue) as the C-terminal amino acid residue.

5. The peptide of claim 1 or 2, wherein the peptide comprises the amino acid sequence of SEQ ID NO:3, optionally wherein the peptide further comprises a linking amino acid residue (e.g., a cysteine residue or lysine residue) as the C-terminal amino acid residue.

6. The peptide of claim 1 or 2, wherein the peptide comprises the amino acid sequence of SEQ ID NO:4, optionally wherein the peptide further comprises a linking amino acid residue (e.g., a cysteine residue or lysine residue) as the C-terminal amino acid residue.

7. The peptide of claim 1 or 2, wherein the peptide comprises the amino acid sequence of SEQ ID NO:5, optionally wherein the peptide further comprises a linking amino acid residue (e.g., a cysteine residue or lysine residue) as the C-terminal amino acid residue.

8. The peptide of claim 1 or 2, wherein the peptide comprises the amino acid sequence of SEQ ID NO:6, optionally wherein the peptide further comprises a linking amino acid residue (e.g., a cysteine residue or lysine residue) as the C-terminal amino acid residue.

9. The peptide of claim 1 or 2, wherein the peptide comprises the amino acid sequence of SEQ ID NO:7, optionally wherein the peptide further comprises a linking amino acid residue (e.g., a cysteine residue or lysine residue) as the C-terminal amino acid residue.

10. The peptide of claim 1 or 2, wherein the peptide comprises the amino acid sequence of SEQ ID NO:8, optionally wherein the peptide further comprises a linking amino acid residue (e.g., a cysteine residue or lysine residue) as the C-terminal amino acid residue.

11. The peptide of claim 1 or 2, wherein the peptide comprises the amino acid sequence of SEQ ID NO:9, optionally wherein the peptide further comprises a linking amino acid residue (e.g., a cysteine residue or lysine residue) as the C-terminal amino acid residue.

12. The peptide of claim 1 or 2, wherein the peptide comprises the amino acid sequence of SEQ ID NO:10, optionally wherein the peptide further comprises a linking amino acid residue (e.g., a cysteine residue or lysine residue) as the C-terminal amino acid residue.

13. The peptide of claim 1 or 2, wherein the peptide comprises the amino acid sequence of SEQ ID NO:11, optionally wherein the peptide further comprises a linking amino acid residue (e.g., a cysteine residue or lysine residue) as the C-terminal amino acid residue.

14. The peptide of claim 1 or 2, wherein the peptide comprises the amino acid sequence of SEQ ID NO:12, optionally wherein the peptide further comprises a linking amino acid residue (e.g., a cysteine residue or lysine residue) as the C-terminal amino acid residue.

15. The peptide of claim 1 or 2, wherein the peptide comprises the amino acid sequence of SEQ ID NO:13, optionally wherein the peptide further comprises a linking amino acid residue (e.g., a cysteine residue or lysine residue) as the C-terminal amino acid residue.

16. The peptide of claim 1 or 2, wherein the peptide comprises the amino acid sequence of SEQ ID NO:14, optionally wherein the peptide further comprises a linking amino acid residue (e.g., a cysteine residue or lysine residue) as the C-terminal amino acid residue.

17. The peptide of claim 1 or 2, wherein the peptide comprises the amino acid sequence of SEQ ID NO:15, optionally wherein the peptide further comprises a linking amino acid residue (e.g., a cysteine residue or lysine residue) as the C-terminal amino acid residue.

18. The peptide of claim 1 or 2, wherein the peptide comprises the amino acid sequence of SEQ ID NO:16, optionally wherein the peptide further comprises a linking amino acid residue (e.g., a cysteine residue or lysine residue) as the C-terminal amino acid residue.

19. The peptide of claim 1 or 2, wherein the peptide comprises the amino acid sequence of SEQ ID NO:17, optionally wherein the peptide further comprises a linking amino acid residue (e.g., a cysteine residue or lysine residue) as the C-terminal amino acid residue.

20. The peptide of any one of claims 1-19, wherein the peptide binds an immunoglobulin (e.g., a polyclonal and/or monoclonal antibody) or fragment thereof, optionally wherein the peptide binds the Fc portion of the immunoglobulin or fragment thereof.

21. The peptide of claim 20, wherein the immunoglobulin or fragment thereof is one or more selected from human IgG (e.g., IgG₁, IgG₂, IgG₃, and/or IgG₄), IgA, IgE, IgD, and IgM.

22. The peptide of any one of claims 20-21, wherein the immunoglobulin or fragment thereof is one or more selected from a non-human mammal (e.g., mouse, rat, rabbit, hamster, horse, donkey, cow, goat, sheep, llama, camel, alpaca, etc.) IgG, IgA, and IgM.

23. The peptide of any one of claims 20-22, wherein the immunoglobulin or fragment thereof is avian (e.g., chicken, turkey, etc.) IgY.

24. The peptide of any one of claims 1-23, further comprising a detectable moiety (e.g., a fluorescent molecule, a chemiluminescent molecule, a radioisotope, a chromogenic substrate, etc.).

25. The peptide of any one of claims 1-24, wherein the peptide is bound to a solid support (e.g., a chromatographic resin, a membrane, a biosensor, a microplate, a fiber, a nanoparticle, a microparticle, or a channel in a microfluidic device), optionally wherein the peptide is bound to the solid support via a linking group (e.g., the side chain group of the linking amino acid residue).

26. An article comprising a solid support (e.g., a chromatographic resin, a membrane, a biosensor, a microplate, a fiber, a nanoparticle, a microparticle, or a channel in a microfluidic device) and the peptide of any one of claims 1-24, optionally wherein the peptide is covalently bound to the solid support (e.g., via the side chain group of the linking amino acid residue).

27. The article of claim 26, wherein the article is an affinity adsorbent.

28. The article of claim 26 or 27, wherein the article is reusable.

29. The article of any one of claims 26-28, wherein the peptide is present at a density in a range of about 0.01, 0.02, 0.05, 0.1, 0.15, or 0.2 mmol of the peptide per mg of the solid support to about 0.25, 0.3, 0.35, 0.4, 0.45, 0.5, 0.55, 0.6, 0.65, 0.7, 0.75, or 0.8 mmol of the peptide per mg of the solid support (mmol/mg).

30. A method of detecting an immunoglobulin or fragment thereof present in a sample, the method comprising:
contacting the sample and the peptide of any one of claims 1-25 or article of any one of claims 26-29 under suitable conditions wherein the peptide binds the immunoglobulin or fragment thereof to provide an peptide-bound immunoglobulin; and
detecting the peptide and/or optionally detecting the detectable moiety, thereby detecting the immunoglobulin or fragment thereof

31. The method of claim 30, further comprising releasing the immunoglobulin or fragment thereof from the peptide and/or article.

32. A method of purifying an immunoglobulin or fragment thereof present in a sample, comprising:
contacting the sample and the peptide of any one of claims 1-25 or article of any one of claims 26-29 under suitable conditions wherein the peptide binds the immunoglobulin or fragment thereof to provide a peptide-bound immunoglobulin; and
releasing the immunoglobulin or fragment thereof from the peptide and/or article, thereby purifying the immunoglobulin or fragment thereof from the sample.

33. The method of any one of claims 30-32, further comprising, prior to releasing the immunoglobulin or fragment thereof from the peptide and/or article, washing the peptide-bound immunoglobulin.

34. The method of any one of claims 30-33, further comprising repeating the contacting step, washing step, and/or the releasing step one or more times, optionally wherein the article is reusable.

35. The method of any one of claims 31-34, wherein the releasing step provides at least 80% (e.g., at least 80, 85, 90, 95, 96, 97, 98, 99, or 100% or any value or range therein) purity of the immunoglobulin or fragment thereof

36. The method of any one of claims 30-35, wherein the sample is from a cell culture fluid (e.g., supernatant), a plant extract, human plasma, transgenic milk, and/or feedstock.

37. The method of any one of claims 30-36, wherein the method provides a host cell protein (HCP) logarithmic removal value (LRV) of at least 2.0, 2.1, 2.2., 2.3, 2.4, 2.5, 2.6, 2.7, 2.8, 2.9, 3.0, 3.1, 3.2, 3.3, 3.4, 3.5, or more as measured by a HCP-specific quantification assay, optionally wherein the method provides a HCP LRV of at least 2.5.
